(19) United States
(12) Patent Application Publication
(10) Pub. No.: US 2022/0364179 A1
(43) Pub. Date: Nov. 17, 2022

(54) METHODS AND COMPOSITIONS FOR ASSESSING AND PREDICTING THERAPEUTIC RESPONSE

(71) Applicant: Lankenau Institute for Medical Research, Wynnewood, PA (US)

(72) Inventor: George C. Prendergast, Penn Valley, PA (US)

(21) Appl. No.: 17/642,108

(22) PCT Filed: Sep. 14, 2020
(86) PCT No.: PCT/US2020/050724

§ 371 Date: Mar. 10, 2022

Related U.S. Application Data
(60) Provisional application No. 62/899,730, filed on Sep. 12, 2019.

(51) Int. Cl. C12Q 1/6886; G01N 33/574

(52) U.S. Cl. CPC C12Q 1/6886; G01N 33/574

(57) ABSTRACT

A method of predicting the responsiveness of a subject having a disease to a treatment with an inhibitor of Indoleamine 2,3-dioxygenase 2 (IDO), an inhibitor of tryptophan 2,3-dioxygenase (TDO), or an inhibitor of the IDO/TDO pathway and/or an additional therapy comprises performing a genotype assay to determine the presence, absence or mutation of the Indoleamine 2,3-dioxygenase 2 (ID02) gene at the single nucleotide polymorphism (SNP) site rs4503084 and the SNP site rs10109853.

### CROSS-REFERENCE TO RELATED APPLICATIONS

This application claims the benefit of the priority of U.S. Provisional Patent Application No. 62/899,730, filed Sep.12, 2019, which application is incorporated herein by reference.

### STATEMENT REGARDING FEDERALLY SPONSORED RESEARCH OR DEVELOPMENT

This invention was made with government support under grant number R01 CA191191 awarded by the National Institutes of Health. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “MLH112PCT_ST25.txt ” is 5 kb in size and is dated Sep. 11, 2020.

### BACKGROUND OF THE INVENTION

Indoximod (C₁₂H₁₄N₂O₂or 1-methyl-tryptophan or D-1MT) is a small molecule inhibitor of the Indoleamine 2,3-dioxygenase (IDO) pathway. Indoximod and other inhibitors of IDO or of tryptophan 2,3-dioxygenase (TDO) are in development as a new class of targeted drug therapy. Cancer clinical trials of indoximod and other IDO small molecule inhibitors suggest that they act as immunometabolic adjuvants to safely improve the efficacy of chemotherapy, radiotherapy, chemoradiotherapy, cancer vaccines, and immune checkpoint therapies. Additionally, preclinical studies indicate that indoximod and IDO inhibitors may also improve the treatment of retinopathies, certain chronic infections and/or certain inflammatory diseases (e.g. autoimmune conditions such as rheumatoid arthritis). In clinical trials there is considerable variability in patient responses to treatment with indoximod or other IDO inhibitors.

No biomarker or test is known that identifies the specific individuals most likely to benefit clinically from treatment with these drugs. Neither IDO1 expression status nor IDO2 expression status alone appears to be predictive. IDO2 genotype varies naturally in human populations. In a mouse model of the autoimmune disease rheumatoid arthritis, deletion of the IDO2 gene was reported to ablate therapeutic responses to indoximod (Merlo et al. 2014 J. Immunol. 192, 2082-2090). Conversely, in a study of the autoimmune disease multiple sclerosis, inactive IDO2 genetic configurations were not associated with any change in disease incidence or progression (Agliardi et al. 2017 J. Neurol. Sci. 377, 31-34).

### SUMMARY OF THE INVENTION

Methods are disclosed herein that use IDO2 genetic variants in subjects as biomarkers for predicting therapeutic responses to indoximod or other IDO inhibitors as well as for predicting cancer progression, among other uses.

In one aspect, a method is provided for predicting the responsiveness of a subject having a disease to a combined treatment with an inhibitor of Indoleamine 2,3-dioxygenase 2 (IDO), an inhibitor of tryptophan 2,3-dioxygenase (TDO), or an inhibitor of the IDO/TDO pathway and a second therapy. The method involves performing a genotype assay to determine the presence, absence or mutation of the Indoleamine 2,3-dioxygenase 2 (IDO2) gene at the single nucleotide polymorphism (SNP) site rs4503084 and the SNP site rs10109853. The occurrence of mutations at one or both SNP sites to a single allele or both alleles impacts activation of the IDO2 and further indicates the type of response the subject is likely to exhibit to a variety of drug regimens and therapies.

In another aspect, a method of assessing the risk of a subject for the onset or progression of cancer comprises performing a genotype assay to determine the presence, absence or mutation of the Indoleamine 2,3-dioxygenase 2 (IDO2) gene at the single nucleotide polymorphism (SNP) site rs4503084 and the SNP site rs10109853. The presence of a mutation at one or both said SNP sites that inactivates the IDO2 activity of both alleles indicates that the subject has a decreased risk of cancer onset.

Still other aspects and advantages and methods employing the test for correlation of the genotype of the IDO2 in the subject's DNA are described further in the following detailed description of the preferred embodiments thereof.

### BRIEF DESCRIPTION OF THE DRAWINGS

FIG. 1 shows that IDO2-deficient mice resist the development of KRAS-induced pancreatic ductal adenocarcinoma (PDAC) in a series of flow cytometric graphs. Total leukocytes (CD45+) obtained from dissociated pancreata from KrasWT or KrasG12D mice on either an Ido2+/+ of Ido2−/− background were analyzed by flow cytometry for specific immune cell subsets (macrophages, dendritic cells, neutrophils, helper T cells or cytotoxic T cells) as indicated. Results from evaluable samples are plotted together with the means±SE (N≥4). Representative histologies (shown as FIG. 1B of ref 51,incorporated by reference herein) of pancreatic tissue were collected at necropsy. H&E-stained tissues obtained from ♀ mice at necropsy diagnosed as moderately differentiated ductal adenocarcinoma (+/+) or PanIN1-3 exhibiting small duct proliferation only (−/−).

FIG. 2. IDO2 genotype and histologic neutrophil/lymphocyte ratio (NLR) are determined from evaluable cases from the TCGA and TJUH cohorts that were subjected to histopathologic analysis of H&E-stained specimens to determine NLRs (TCGA N=95 panel 1, TJUH N=43 panel 2). NLR scores were segregated according to IDO2 functional genotype status using the IDO2 genotype functionality definitions of Table 5 below. Representative images are scored for this analysis from the TJUH cohort (not shown).

FIGS. 3A and 3B show that IDO2-deficient genotype in human PDAC is associated with improved DFS of patients who receive radiotherapy. DFS analyses of the pooled TCGA-PAAD and TJUH cohorts stratified by IDO2 functional genotype. FIG. 3A is a Kaplan-Meier analysis of all cases (N=168). FIG. 3B is a Kaplan-Meier analysis of cases with evidence of microscopic tumor involvement (R1 resection margins, N=61).

FIGS. 4A-4D show that IDO2-deficient genotype in human PDAC is associated with improved DFS of patients who receive radiotherapy. DFS analyses of the pooled TCGA-PAAD and TJUH cohorts are stratified by IDO2 functional genotype. FIG. 4A is a Kaplan-Meier analysis of pooled cases (N=168). FIG. 4B is a Kaplan-Meier analysis of cases receiving radiotherapy (N=54). FIG. 4C is a Kaplan-Meier analysis of cases not receiving radiotherapy (N=77). FIG. 4D is a Cox multivariate hazard analysis (N=124).

FIG. 5 is a schematic of the normal human IDO2 SNP distribution. Symbols: WT means wild-type allele, MUT means mutated allele; AFR means African population; AMR means American population, EAS means Eurasian population; EUR means European population and SAS means South American populations.

FIGS. 6A-6D provide raw data used in Example 3.

FIGS. 7A-7B provide raw data used in Example 5.

### DETAILED DESCRIPTION

Heritable genetic variations that affect the inflammatory tumor microenvironment can ultimately affect cancer susceptibility and clinical outcomes. Methods are disclosed that use IDO2 genetic variants in subjects as biomarkers for predicting which human subjects are likely to demonstrate therapeutic responsiveness to treatment with to indoximod, IDO/TDO inhibitors or IDO/TDO pathway inhibitors, as well as additional therapies.

### A. Components and Definitions of the Methods Described

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

The terms “a” or “an” refers to one or more. For example, “an expression cassette” is understood to represent one or more such cassettes. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited. As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP. sctn. 2111.03. Thus, the term “consisting essentially of” when used in a claim of this invention is not intended to be interpreted to be equivalent to “comprising.”

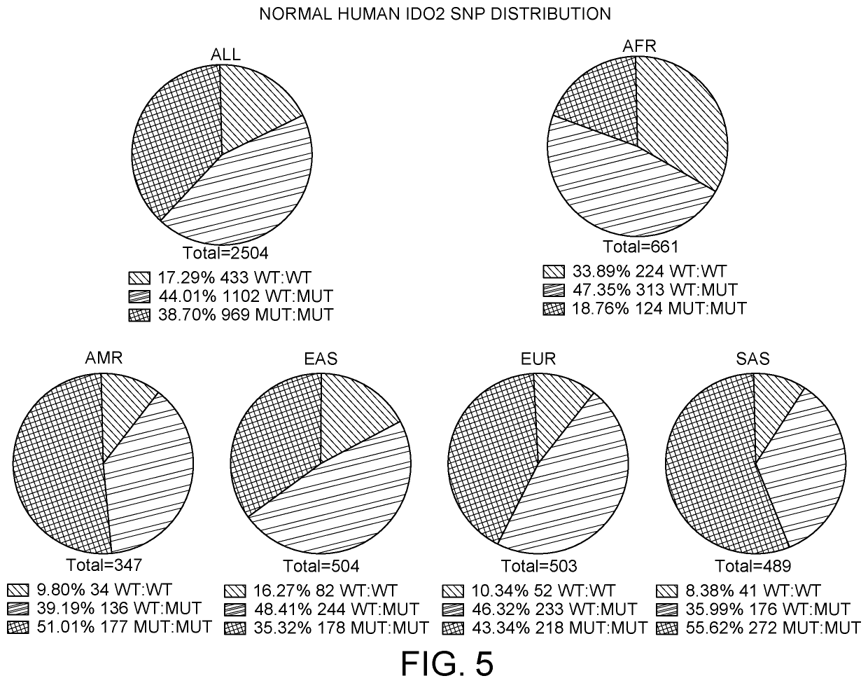

“IDO-2” nucleic acid sequences and encoded amino acid sequences are described publicly in U.S. Pat. No. 8,058,416 and in NCBI database NM 194294.2 and NP919270.2. U.S. Pat. No. 8,058,416 describes the protein coding sequences of IDO2 found in the genomic DNA, particular the coding sequence of human IDO2 which is duplicated herein as SEQ ID NO: 5. A unique feature of IDO2 in humans is the high prevalence of two inactivating single-nucleotide polymorphisms (SNP). The two single-nucleotide polymorphisms (SNP) in the coding region of human IDO2 have the locations rs4503083 and rs10109853 and decrease enzymatic activity through amino acid substitution at the active site (R248W) or by truncation of the enzyme (Y359X), respectively. See, e.g., Metz R, et al, Novel tryptophan catabolic enzyme IDO2 is the preferred biochemical target of the antitumor indoleamine 2,3-dioxygenase inhibitory compound D-1-methyl-tryptophan. Cancer Res 2007; 67:7082-7. Notably, while both SNPs are quite prevalent in human populations (see FIG. 5), their clinical significance has remained undefined

For the IDO2 genotype nomenclature as used herein, “+” designates a wild-type allele and “p” designates an inactive SNP allele. Homozygous WT is +/+; Heterozygous is +/p (in which the second allele is one of the two enzymatically inactive alleles), and Homozygous inactive is p/p (in which the subject has two copies of either inactive allele). The SNP polymorphisms for each of the two alleles are shown in the third and fourth column of Table 1 below in relation to the IDO2 enzymatic activity associated with the genotype. For example, C/C means a cytosine base at both positions on alleles at the site of the SNP; C/T means a cytosine and a thymine at the positions on the alleles at the site of the SNP. These symbols are used throughout the specification and in the examples below.

TABLE 1

| | + = WT allele; | | |
|---|---|---|---|
| | +/p = Het; | SNP allele 1 | SNP allele 2 |
| | p/p = homozygous | rs4503083 | rs10109853 |
| Functional Genotype | inactive | (R325W) | (Y346X) |
| Homozygous Active | +/+ | C/C | T/T |
| Heterozygous | +/p | C/C | T/A |
| Heterozygous or | +/p or p/p | C/T | T/A |
| Homozygous Inactive | | | |
| Homozygous Inactive | p/p | T/T | ANY |
| Homozygous Inactive | p/p | ANY | A/A |

Techniques that can be used to identify single nucleotide polymorphisms (SNPs) useful in the methods described herein include, but are not limited to, whole genome exome sequencing (using next generation sequencing technology, i.e., NGS), targeted allelic sequencing, which focuses on the target genes instead of the whole genome, by generating amplicons by PCR, and/or techniques based on Taqman Sanger sequencing, which is equivalent to the targeted allelic sequencing, but does not use NGS. All techniques are valid to determine and identify the SNPs discussed herein.

By “mammalian subject”, “patient” or “subject” as used herein means a mammalian animal, including a human, a veterinary or farm animal, a domestic animal or pet, and animals normally used for clinical research. More specifically, the subject of these methods and compositions is a human In one embodiment, the subject has a cancer.

A “subject in need thereof” or “a subject in need of” is a subject known to have or is suspected of having or developing a disease for which treatment with IDO/TDO inhibitors or IDO pathway inhibitors associated with radiation therapy and/or chemotherapy and/or immunotherapy is contemplated. In particular embodiments, the subject is in need of, is scheduled for, and/or is planning to undergo radiation and/or chemotherapy and/or immunotherapy with adjuvant treatment with IDO/TDO inhibitors or IDO pathway inhibitors, and/or other cancer treatment.

Diseases for which IDO/TDO inhibitors or IDO pathway inhibitors are useful include cancer, chronic infection, autoimmune disease, retinopathy and others suggested in the biomedical literature.

As used herein the term “cancer” refers to or describes the physiological condition in mammals that is typically characterized by unregulated cell growth. In one embodiment, the term “cancer” means any cancer characterized by the presence of a solid tumor. In another embodiment, a cancer is a hematological cancer. When referred to herein, a cancer includes, without limitation, pancreatic cancer or PDAC, melanoma, breast cancer, brain cancer, colon/rectal cancer, lung cancer, ovarian cancer, adrenal cancer, anal cancer, bile duct cancer, bladder cancer, bone cancer, endometrial cancer, esophagus cancer, eye cancer, kidney cancer, laryngeal cancer, liver cancer, head and neck cancer, nasopharyngeal cancer, osteosarcoma, oral cancer, ovarian cancer, prostate cancer, rhabdomyosarcoma, salivary gland cancer, stomach cancer, testicular cancer, thyroid cancer, vaginal cancer, lung cancer, and neuroendocrine cancer, glioblastoma, among others.

“PDAC” refers to pancreatic ductal adenocarcinoma, the main pancreatic cancer.

By “biological sample” or “sample” as referred to herein, is meant cells, tissue and/or fluid containing cells or cellular debris of the subject that can be used to identify the genetic marker profile of the subject, and specifically IDO2 gene sequences. Such samples include, blood, peripheral blood, plasma, saliva, urine, cerebrospinal fluid, tumor biopsy tissue, tears, and other secretions from the subject that contain DNA. In one embodiment, the sample is diluted. In another embodiment, the sample is a concentrated sample.

The terms “increased risk” and “decreased risk” or “likelihood” as used herein define the level of risk that a subject has of responding to therapeutic treatment with the IDO/TDO inhibitors or IDO pathway inhibitors as described herein, as compared to a control subject.

By “responder” as used herein is meant a subject that received an IDO inhibitor and/or other therapy in the course of a study.

By “drug regimen” as used in the methods described herein is generally meant either the combined or sequential administration of 1 or 2 different IDO/TDO inhibitor drugs in combination or sequentially with radiation therapy, chemotherapy or immunotherapy or the combined or sequential administration of 3 to 10 different IDO/TDO inhibitor/IDO pathway inhibitor drugs.

By “IDO/TDO inhibitors and/or IDO pathway inhibitors includes, without limitation, indoximod, Epacadostat, BMS-98605, Navoximod, Choroquine, Acyclovir, PF-06840003, IOM2983, RG-70099 and CB548, among others known in the scientific literature. It is anticipated that any newly identified IDO/TDO inhibitors and/or IDO pathway inhibitors can be used similarly with respect to this assay described herein.

By immunotherapy agents as used herein includes without limitation PD1 checkpoint therapy including drugs such as Keytruda® or dendritic cell therapy including drugs such as Provenge®.

“Chemotherapeutic agents” as used herein includes without limitation, chemotherapeutic agents include alkylating agents such as thiotepa and cyclosphosphamide; alkyl sulfonates such as busulfan, improsulfan and piposulfan; aziridines such as benzodopa, carboquone, meturedopa, and urcedopa; ethylenimines and methylamelamines including altretamine, triethylenemelamine, triethylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as elodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY 17018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above “PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1 ; and PDCD1L2, PDL2, B7-DC, Btdc and CD273 for PD-L2. Examples of mAbs that bind to human PD-1, are described in U.S. Pat. Nos. 7,488,802, 7,521,051, 8,008,449, 8,354,509, 8,168,757, WO2004/004771, WO2004/072286, WO2004/056875, and US2011/0271358. Specific anti-human PD-1 mAbs useful as the PD-1 antagonist include: MK-3475, a humanized IgG4 mAb with the structure described in WHO Drug Information, Vol. 27, No. 2, pages 161-162 (2013), nivolumab (BMS-936558), a human IgG4 mAb with the structure described in WHO Drug Information, Vol. 27, No. 1, pages 68-69 (2013); the humanized antibodies h409A11, h409A16 and h409A17, which are described in WO2008/156712, and AMP-514, which is being developed by MedImmune. An exemplary anti PD-1 is the antibody marketed as KEYTRUDA.

By radiotherapy as used herein is meant treatment that uses high doses of radiation to shrink tumors or kill cancers cells. Radiotherapy includes both external beam radiation therapy, such as photon beam therapy and 3-dimensional conformational therapy as well as internal radiation therapy, e.g., brachytherapy in which seeds or capsules containing a radioactive element are implanted or systemic radiation therapy,

### B. Methods for Diagnosis and Prognosis Based Upon the IDO2 Genotype

In one aspect a method of predicting the responsiveness of a subject having a disease to a therapeutic treatment regimen comprises performing a genotype assay to determine the presence, absence or mutation of the Indoleamine 2,3-dioxygenase 2 (IDO2) gene at the single nucleotide polymorphism (SNP) site rs4503084 and the SNP site rs10109853. The variant genotypes provided by the two SNP sites can provide several correlations that are predictive of disease occurrence, progression and response to treatment regimens. In one embodiment, the genotype of the subject can indicate a greater responsiveness to radiotherapies, chemotherapies or immunotherapies. In another embodiment, the genotype of the subject can indicate a greater responsiveness to combined treatment with an inhibitor of Indoleamine 2,3-dioxygenase 2 (IDO), an inhibitor of tryptophan 2,3-dioxygenase (TDO), or an inhibitor of the IDO/TDO pathway. In still another embodiment, the variant genotypes can indicate a greater responsiveness to combined treatment with an inhibitor of Indoleamine 2,3-dioxygenase 2 (IDO), an inhibitor of tryptophan 2,3-dioxygenase (TDO), or an inhibitor of the IDO/TDO pathway with a second therapy (e.g., radiotherapy, immunotherapy or chemotherapy).

For example, as shown in Table 1, the occurrence of the wild-type nucleotides on both alleles at each SNP provides for a homozygous (+/+) fully active IDO2. Persons with this wildtype genotype have been found to be more susceptible to certain cancer occurrences, such as the pancreatic cancer that is the subject of Example 1 below. The occurrence of a mutation of one or both SNPs in which at least one mutation appears on each IDO2 allele provides for a homozygous (p/p) inactive IDO2. This genotype has been found to correlate with a subject having a positive response to treatment involving radiotherapy.

Still another genotype in which one allele contains the wildtype nucleic acids of both SNPs while the other allele carries a mutation at one or both SNPs, results in a heterozygous genotype (+/p) and can generate partial IDO2 activity. This genotype can indicate that a subject can respond to a therapeutic regimen, but not as favorably or positively as the homozygous active IDO2.

The genotype in which both SNPs are heterozygous can generate either an +/p when both mutations are on the same allele, or a p/p when the single mutation from each of the SNPs is on a different allele. As shown in the Example 3, the p/p genotypes respond less well to the IDO/TDO inhibitors with chemotherapy, but better to the IDO/TDO inhibitors with radiotherapy.

It is anticipated that these genotypes themselves serve as biomarkers for guiding the selection of adjuvant therapies or combined therapies for subjects with a disease susceptible to these therapies. In one embodiment, it is the combination of therapies for which the genotypes provide an indicate of patient responsiveness.

The performance of the genotype assay involves obtaining DNA from a biological sample of said subject; and contacting the DNA sample from the subject with reagents to determine the presence, absence or a mutation at the following wildtype alleles:
i. CC at single nucleotide polymorphism (SNP) site rs4503084
ii. TT at SNP site rs10109853.
   Where the primary therapy or second therapy (i.e., a therapy that is administered with or in sequence with IDO/TDO inhibitors or pathway inhibitors) is a radiotherapy, either external beam or internal radiotherapy, the genotype p/p provides a more promising biomarker of positive response. Where the primary therapy or second therapy (i.e., a therapy that is administered with or in sequence with IDO/TDO inhibitors or pathway inhibitors) is immunotherapy, such as the administration of an anti-PD1 composition or dendritic cell therapy, it is anticipated that the genotype of the IDO 2 SNPs will also indicate which subject will be predicted to fare better. Where the primary therapy or second therapy (i.e., a therapy that is administered with or in sequence with IDO/TDO inhibitors or pathway inhibitors) is chemotherapy, the preliminary data supports that the subject with a genotype +/+ is expected to respond better. Still other correlations are anticipated to be uncovered using this genotype test.

While the examples below demonstrate use of the genotype biomarker with a pancreatic ductal adenocarcinoma (PDAC), a brain cancer, and melanoma, it is anticipated that this IDO2 genotype will be predictive for responses to other cancers as well as for determining susceptibility to treatment with an IDO inhibitor, including: breast cancer, brain cancer, colon/rectal cancer, and the other cancers identified above. Similarly, for other diseases for which the IDO/TDO inhibitors are currently useful, e.g., chronic infection, an autoimmune disease, or retinopathy, among others, the genotype is also anticipated to function similarly as a biomarker for therapeutic responsiveness.

The examples below also demonstrate combinations of therapies for which the genotypes provide a predictive result indicating which subjects would be more benefited by different therapeutic regimens.

The present invention is a simple genetic test that defines a genetic configuration associated with favorable clinical responses to IDO inhibitors or IDO pathway inhibitors. The invention solves the problem of a lack of a biomarker to focus clinical development of indoximod and other IDO inhibitors or IDO pathway inhibitors. Further, the invention solves the need for a companion test to direct drug treatment only to those patients capable of responding successfully to treatment. The methods described herein are also useful as genetic tests for clinical development and administration of indoximod and other IDO inhibitors or IDO pathway inhibitors and can be used to identify patients capable of responding to these drugs, based on their IDO2 genotype.

In one embodiment, the IDO genotype variants described herein can be used as a biomarker to enable targeted patient recruitment to clinical trials of indoximod, IDO/TDO inhibitors or IDO/TDO pathway inhibitors. The methods can be used to identify patients most likely to favorably respond clinically to indoximod, IDO/TDO inhibitors or IDO/TDO pathway inhibitors as well as to additional or combination therapies such as radiotherapy, immunotherapy and chemotherapy.

As disclosed in the Examples below, evidence indicates that IDO2, a positive modifier in inflammatory disease models, is frequently upregulated in pancreatic ductal adenocarcinoma (PDAC). In seeking to address whether genetic loss of IDO2 may influence PDAC development and responsiveness to treatment, we conducted a set of initial preclinical and clinical studies. Example 1 reports on a clinical trial that showed that human IDO2 gene variants are associated with extended overall survival in pancreatic cancer patients who receive radiotherapy. More specifically an inactive IDO2 genetic configuration is associated with extended overall survival if adjuvant radiotherapy was administered. This study was first published on Sep. 28, 2018 and is described in more detail in Nevler et al. 2019 Clin. Cancer Res. 25, 724-734 (ref 51), incorporated by reference.

Transgenic Ido2 +/+ and Ido2 −/− mice in which oncogenic KRAS is activated in pancreatic epithelial cells were evaluated for PDAC. Tumor development was notably decreased in the Ido2 −/− mice (30% vs. 10%, P<0.05). For the clinical study, a patient dataset (N=200) was evaluated for the two IDO2-inactivating SNPs together with histologic, RNA expression, and clinical survival data. Biallelic occurrence of either of the two IDO2-inactivating SNPs was significantly associated with markedly improved disease-free survival in response to adjuvant radiotherapy (P<0.01), a treatment modality that has been highly debated due to its variable efficacy.

In a related study, we took advantage of laboratory evidence that IDO2 activity can be ablated by administering low-dose chloroquine, an anti-inflammatory modality that has been evaluated in solid tumors including PDAC (although not as a radiosensitizer). Briefly, in patients with brain metastases who received whole brain radiotherapy (N=20), administration of low-dose chloroquine in continuous cycles one week before and during standard of care irradiation produced a trend in improvement of survival outcomes (P=0.07).

Lastly, in an ongoing study we took advantage of laboratory evidence that IDO2 can be targeted by indoximod, an IDO pathway inhibitor studied in combination with gemcitabine in PDAC patients (N=143). In the initial cohort of patients analyzed from this trial, stratifying outcomes to IDO2 genotype yielded a trend in improved therapeutic responses in patients harboring functional IDO2 alleles. See Example 3.

In yet another aspect, a method is described for assessing the risk of a subject for the onset or progression of cancer by performing a genotype assay to determine the presence, absence or mutation of the Indoleamine 2,3-dioxygenase 2 (IDO2) gene at the single nucleotide polymorphism (SNP) site rs4503084 and the SNP site rs10109853, wherein the presence of a mutation at one or both said SNP sites that inactivates the IDO2 activity of both alleles indicates that said subject has a decreased risk of cancer onset.

In one embodiment, this method was demonstrated for pancreatic ductal adenocarcinoma (PDAC)—see Example 1. However, it is further anticipated to be a useful biomarker to predict onset of other cancers. It is anticipated that the data of Example 1 can be extrapolated to other cancers, such as those listed above.

Still another embodiment of these diagnostic and prognostic methods involves performing the diagnostic method and genotyping steps, following by a treatment step of administering to the subject an effective amount of the compounds and therapeutics for which the test indicated that the subject was likely to be susceptible.

Our findings support the evaluation of patient IDO2 genotype as a prognostic tool in patients with PDAC and other metastatic solid tumors, with the potential to assist decision making in the care of patients who stand to benefit most from adjuvant radiotherapy, low-dose chloroquine or indoximod, among other therapies.

### EXAMPLE 1—PDAC STUDY

In this study, we have obtained genetic evidence supporting IDO2's relevance to PDAC tumorigenesis using a Kras-driven PDAC mouse model (17) in which Ido2 was genetically targeted for deletion, in conjunction with an analysis of the prevalence of the two IDO2-inactivating SNPs in PDAC patients. Based on these findings, we performed retrospective analyses of treatment outcomes for surgically resected PDAC patients based on their IDO2 genotype status. For the subset of patients who had received adjuvant radiotherapy during the course of treatment, these analyses uncovered a significant association between IDO2-deficient status and improved disease-free survival, a finding with potential ramifications for informing future treatment decisions for this intractable disease.

### A. Methods and Materials

Mouse husbandry and histopathology: IDO2-nullizygous mice and the genetically defined KC mouse model of KRAS-induced PDAC on a common C57BL/6J background strain have been described (11, 17). PDX-1-cre; LSL-KrasG12D transgenic mice (KC mice) develop pancreatic intraepithelial neoplasias (PanlNs) with complete penetrance along with sporadic focal pancreatic carcinomas with reduced penetrance due to PDX1-cre-mediated activation of the latent oncogenic KrasG12D allele in pancreatic progenitor cells (17). These KRAS-induced lesions elicit a robust inflammatory response including B-cell contributions (18) where IDO2 may act to influence the tissue microenvironment (10). To investigate this hypothesis in an autochthonous pancreatic tumor setting, we introduced Ido2−/− (Ido2-nullizygous) alleles (11) into the LSL-KrasG12D mouse strain. Mice were interbred to generate Ido2+/+ and Ido2−/− KC siblings in which KRAS is activated with similar kinetics for longitudinal comparisons of disease initiation and progression for 11 to 13 months duration. Two independent cohorts were generated and analyzed. Histologic analysis of pancreatic lesions was conducted by standard methods as previously described (17, 19).

Flow cytometry analysis of infiltrating immune cells in mouse pancreata: Single-cell suspensions were prepared from resected pancreata using a gentleMACS Octo Dissociator with the Tumor Dissociation Kit as per the manufacturers' instructions. Levels of the following cell-surface markers were directly measured by flow cytometry on a BD FACSCanto (BD Biosciences) in two separate groups as noted and analyzed using FlowJo Software (Tree Star). Group 1: CD45 (APC; BioLegend), CD11b (PE/Cy7; BioLegend), Gr1(PerCP; BioLegend), CD11c (PE; BioLegend), F4/80 (Alexa Fluor 488; BioLegend), Fixable Viability Dye (eFluor 780; eBioscience), Group 2: CD45 (APC; BioLegend), Thy1.2 (Alexa Fluor 488; BioLegend), IgMa (PE; BD Pharmingen), CD4 (PE/Cy7; BioLegend), CD8a (PerCP; BD Pharmingen), Fixable Viability Dye (eFluor 780; eBioscience).

Preparation and genotyping of patient tissue specimens: The study was conducted in accordance with the ethical guidelines of the Belmont Report with a statement of informed written consent obtained from each subject as appropriate. From the IRB-approved Thomas Jefferson University Hospital data set (TJUH data set, all patients in the cohort have given their informed consent) genomic DNA from surgically resected pancreatic tissue specimens (normal and tumor tissues) was extracted using the DNAeasy Blood and Tissue Kit genomic DNA purification kit (Qiagen Inc.). DNA fragments containing the IDO2-coding region polymorphisms rs4503083 (Exon 11) and rs10109853 (Exon 9) were amplified by PCR as described previously (IDO2 oligonucleotide primers R248W FWD and R248W REV; Y359X FWD and Y359X REV; ref. 14), as detailed in Table 4. PCR reactions were performed in 25 μL using 100 ng of gDNA, 0.5 μg/μL of Taq polymerase (Affymetrix), 1 μL of 10 μmol/L oligonucleotide primers (forward and reverse), 2.5 μL of 10X PCR buffer (Affymetrix), and 0.5 μL 10 mmol/L dNTP Mix (Affymetrix). PCR reaction products were purified using a commercial PCR purification kit (Qiagen Inc.). Each PCR reaction was examined by gel electrophoresis on a 0.75% DNA agarose gel before Sanger sequencing by a commercial provider (GenScript Inc.) using the DNA oligonucleotide primers mentioned above (14). Genotyping steps were blinded to clinical data and familial-sporadic patient status. IDO2 genotype in patients was determined by chromatogram (14).

Patient tissue specimens: Two pancreatic cancer patient sets were used in this study as described below. Demographic and histologic data are summarized in Table 2.

TABLE 2

| | | TJUH | TCGA | |
|---|---|---|---|---|
| Patient and tissue | | (N = 77) | (N = 123) | |
| specimen characteristics | | N (%) | N (%) | P |
| Gender | Male | 38 (49%) | 68 (55%) | NS |
| | Female | 39 (51%) | 55 (45%) | |
| Race | White/European | 67 (87%) | 109 (89%) | NS |
| | African American | 5 (7%) | 5 (4%) | |
| | Asian | 3 (4%) | 6 (5%) | |
| | Other | 2 (2%) | 3 (2%) | |
| Age (years) | | 66.9 (±9.3) | 65.3 (±11.2) | NS |
| rs4503083 | Y359/Y359 | 52 (68%) | 84 (68%) | |
| (Y359X) | Y359/359X | 18 (23%) | 37 (30%) | |
| | 359X/359X | 7 (9%) | 2 (2%) | |
| | R248/R248 | 23 (30%) | 58 (47%) | |
| rs10109853 | R248/248W | 39 (51%) | 46 (37%) | |
| (R248W) | 248W/248W | 15 (19%) | 19 (16%) | |
| Familial PDA | | 14 (18%) | N/A | |
| Histopathology | Tumor size (cm) | 3.6 (±1.6) | 3.9 (±1.6)^{a} | NS |
| | Poor tumor grade | 20 (26%) | 36 (29.3%) | NS |
| | Metastatic | 57 (74.0%) | 94 (76.4%) | NS |
| | lymph nodes | | | |
| | Lymph node ratio | 0.19 (±0.22) | 0.08 (±0.28) | NS |
| | Positive | 23 (29.9%) | 50 (43.5%)^{a} | NS |
| | surgical margins | | | |
| | Perineural invasion | 64 (83.1%) | N/A | |

TCGA-PAAD data set: The cohort used for this data set included 123 patients from The Cancer Genome Atlas (TCGA) research network database (20). All patients included in the analysis were diagnosed with histologically confirmed PDAC. The set included demographic data, operative findings, histologic features including percentages of lymphocyte and neutrophil infiltrates, RNA expression data, complete variant genotyping as well as survival and recurrence data.

TJUH resected pancreatic cancer data set: The cohort used for this data set included 77 patients who underwent surgical resection with curative intent at the Thomas Jefferson University Hospital, and we had available tissue for DNA analysis (TJUH, IRB Consented). Patient specimens analyzed included PDAC cases that were familial (n=14, 18%) or sporadic (n=63, 82%) Familial cases were defined as two first-degree family members with PDAC as established in previous reports (21). Medical history, preoperative laboratory tests, surgical and histologic findings, and oncologic follow-up data were recorded from the patients' medical records Familial data were extracted from medical records and from the Jefferson Pancreatic Tumor Registry.

TJUH and TCGA data sets were pooled into a single large data set and screened for duplicates. A unified set containing only PDA patients which underwent primary resection was composed (N=200).

Human tissue histology and neutrophil-lymphocyte ratio (NLR) analysis: In the TJUH data set, slides were reviewed by Thomas Jefferson University pathologists (MC, TV). For slide quantification, hematoxylin and eosin stained sections of formalin-fixed paraffin-embedded tumor from the selected patients were reviewed for pathologic confirmation and the adequacy of tissue quantity and preservation for NLR determination. NLR was derived from the quotient of the absolute neutrophil count and the absolute lymphocyte count. For each sample, NLR was determined in three areas each measuring 0.785 mm2. Final NLRs for each specimen were calculated as the average value of the three areas analyzed. Cases in which no immune cell infiltrates and no tumor cells were found were excluded from the analysis as well as cases with active biliopancreatic sepsis (e.g., cholangitis, acute pancreatitis, peripancreatic abscess, etc.). NLR data of 43 TJUH patients (56%) were available for analysis. In the TCGA data set, hematoxylin and eosin (H&E)-stained sections of snap-frozen OCT embedded tissues were reviewed by TCGA-participating histopathologists for validation as PDAC and analysis of histologic features. Tumor, normal, and stromal components were quantified (in percentages) as well as proportion of immune cells (neutrophils, monocytes, lymphocytes; ref. 22). Slides with no or sparse immune cell infiltrates (lymphocytes %+neutrophils %≤10%) were excluded from the analysis. NLR was calculated for each slide and averaged per patient in cases of multiple patient slides. Cases with no visible neutrophilic infiltration were recorded as 0.01% infiltration to allow subsequent log10 transform and Z-score calculation. NLR data of 56 patients (46%) were available for analysis. Overall, 99 patients were included from the pooled data set.

Statistical analysis: Categorical data were expressed as percentages and continuous data were expressed as mean±standard deviation. For normally distributed continuous variables, a Student t test was used. Variables were assessed for normality of distribution with the Kolmogorov-Smirnov test. Comparisons between genotype groups were performed using Mann-Whitney and Jonckheere-Terpstra (J-T) tests for nonparametric distributions and t tests for normal distributions (23). Categorical data were compared with _{χ}2 test or Fisher exact test. P values of ≤0.05 were defined as significant.

Genotype distribution analysis: Genotype distribution was quantified for all sets as well as separately for familial PDAC cases and sporadic PDAC cases. Genotype distribution of each polymorphism was analyzed for Hardy-Weinberg deviation using _{χ}2 test and Fisher exact test. A genotype distribution set of Utah residents (CEPH) with northern and western ancestry was available from the 1000 Genomes Project to be used as a control for comparison of the PDAC patient sets. Patients were grouped into two categories dependent on IDO2 genotype: homozygous alleles of either R248W (rs4503083) or the Y359X (rs10109853) polymorphism and patients with a double-heterozygous genotype (i.e., WT:R248W or WT:Y359X) were considered IDO2-deficient genotypes, whereas all other combinations were considered as active or partially active IDO2 genotypes. For correlation studies, a three-tier scale (Table 5 below) was used to rate the possible combined R248W/Y359X genotypes in terms of probable IDO2 functionality.

IDO2 genotype correlation with inflammation and NLR: TJUH and TCGA specimens in which histopathologic examination defined a high tumor cellularity (≥50% cellularity) and evidence of inflammatory infiltrate (lymphocyte %+neutrophil %>10%) were included in the analysis. Bivariate nonparametric Spearman test was used to assess correlations between IDO2 functionality grade and histologic NLR scores. In-between group comparisons were performed for the pooled data set with subanalyses for both data sets. Slides were reviewed by TJUH pathologists (M. Curtis and T. Villatoro).

IDO2 genotype correlation with immune expression profile: RNA expression data were collected from the TCGA database using cBioPortal (24, 25). Tissue immune cell counts were imputed from GAPDH-normalized gene-expression levels (based on the Affymetrix 133Plus2 gene-expression set) using the validated MCP-Counts algorithm described previously (26, 27). NLR scores were imputed from the cell count estimates. Estimates and ratios were compared across IDO2 gene functionality grades (fully active genotype, partially active genotype, deficient genotype).

Survival analysis: The primary hypothesis was that IDO2-deficient genotypes conferred a favorable prognostic effect. Kaplan-Meier survival analysis stratified by lymph node metastasis was used with log-rank tests to compare survival according to tumor grade, tumor size, perineural invasion, and IDO2 genotype (deficient vs. active/partially active). Survival data from the TCGA-PAAD cohort was limited due to 40% and 63% censorship rates for overall survival (OS) and disease-free survival (DFS), respectively. Survival data from the TJUH cohort revealed censorship rates of 18% and 19% for OS and DFS, which were suitable for subsequent survival analysis. In order to utilize the full extent of the survival data, we performed the survival analyses on the pooled data set as well as in the separate subsets. To prevent possible misclassification biases from patients who were operated upon expecting early-stage disease but having advanced disease, analyses of DFS excluded cases in which recurrence of the disease was diagnosed before 2 months had elapsed from surgery (i.e., DFS≤2 months). Factors with P<0.2 were subsequently included in a Cox multivariate hazard model and were used to assess the impact of IDO2-deficient genotypes on OS time and DFS time. The model was further optimized by sequential inclusion of statistically relevant factors (P≥0.2) until achievement of a final optimal model fit (P≤0.05). P values≤0.05 were considered statistically significant. Statistical analysis was performed using the Statistical Package for Social Sciences (IBM SPSS, Ver.20, SPSS Inc.).

B. IDO2 Deficiency is Associated with Reduced PDAC Tumor Development in Mutant Kras Transgenic Female Mice and in Later Onset Patients

Based on evidence of frequent overexpression of IDO2 in human PDAC tumors (14), we hypothesized that IDO2 inactivation (i.e., loss-of-function IDO2 alleles) might limit the development of pancreatic cancer. KRAS mutations occur in over 90% of invasive PDAC and are an early oncogenic event (28). The transgenic mouse model Pdx-1-cre; LSL-KrasG12D (KC), with an inducible oncogenic Kras allele that is activated in pancreatic progenitor cells, spontaneously develops pancreatic intraepithelial neoplasia (PanIN) with complete penetrance and PDAC with reduced penetrance (17).

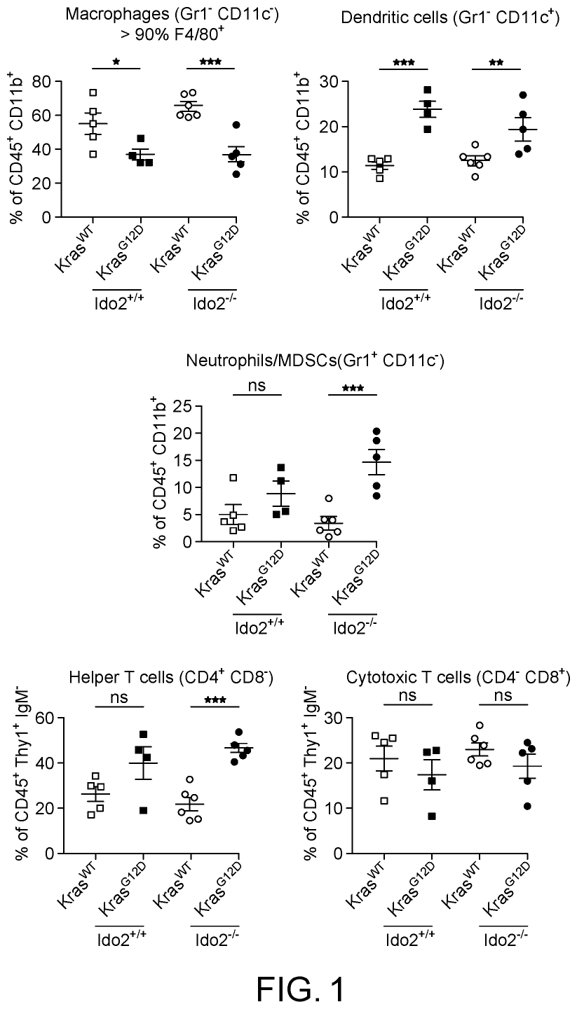

To investigate our hypothesis that IDO2 contributes to pancreatic tumor development in this autochthonous tumor setting, we introduced Ido2−/− (Ido2-nullizygous) alleles into the KC strain through interbreeding (11). Cre-mediated expression of the mutant Kras allele resulted in small duct proliferation in the pancreas regardless of the Ido2 status, along with a decreased frequency of macrophages (CD45+CD11b+Gr1+CD11c−F4/80+) and an increased frequency of dendritic cells (CD45+CD11b+Gr1−CD11c+. See, FIG. 1.

Quantitation of invasive carcinoma diagnosed in KC mice of Ido2 wild-type (+/+) or nullizygous (−/−) genotype at 11 to 13 months of age (lifespan study) is shown in the Table 3 following:

TABLE 3

| All Mice | Invasive PDAC (P = 0.08) | Endpoint Age (wk ± SE) |
|---|---|---|
| Ido2 ^{+/+} | 17/60 (28.3%) | 47.2 ± 2.4 |
| Ido2 ^{-/-} | 7/46 (15.2%) | 47.4 ± 2.6 |
| Female Mice | Invasive PDAC (P = 0.01) | |
| Ido2 ^{+/+} | 10/37 (27.0%) | 44.5 ± 2.1 |
| Ido2 ^{-/-} | 0/24 (0%) | 47.0 ± 4.4 |
| Male Mice | Invasive PDAC (P = NS) | |
| Ido2 ^{+/+} | 7/23 (30.4%) | 51.5 ± 5.1 |
| Ido2 ^{-/-} | 7/22 (31.8%) | 47.9 ± 3.0 |

Increased frequencies of neutrophils/MDSCs (CD45+CD11b+Gr1+CD11c−), and helper T cells (CD45+Th1+IgM−CD4+CD8−) were also associated with mutant Kras expression but were only significant in the mice that also lacked IDO2. No significant differences associated with either Kras or Ido2 status were observed in the frequency of cytotoxic T cells (CD45+Th1+IgM−CD8+CD4−) while the overall number of mature B cells (CD45+Thl−IgM+) was consistently too low to produce meaningful comparisons. Ductal adenocarcinomas were identified in Ido2+/+ KC mice with an overall lifetime incidence of 28% compared with 15% in the Ido2−/− KC mice (P<0.08, FIGS. 1B and C). Unexpectedly, the impact of Ido2 loss on PDAC development in this model was predominantly associated with females, with no ductal adenocarcinomas identified in the 24 female Ido2−/− KC mice while the incidence in Ido2+/+ KC females remained at 30% (P=0.01). Complementing this work, when combining the university (TJUH) data set with The Cancer Genome Atlas research network database (TCGA) data set (n=200), we observed a significant absence of female patients harboring a homozygous R248W (IDO2-deficient) genotype (OR, 0.35; CI 95%, 0.17-0.74; P<0.01).

These observations provide genetic evidence that IDO2 can contribute to the progression of early-stage pancreatic precursor lesions to malignant carcinoma and suggest that IDO2's involvement in this process may be subject to some degree of sexual dimorphism. Similar findings of dimorphism in KC mice have been reported by Chang, et al. (29) showing that female KC mice on a high fat-high calorie diet were less likely to develop PDAC compared with male KC mice (0% vs. 44% and 33% vs. 50%, at 6 and 9 months, respectively). Similar to these findings in the KC experiment, sequencing of the R248W SNP in the TJHU cohort revealed a significant paucity of R248W homozygous cases (IDO2-deficient status) in females with sporadic PDAC compared with the CEU control population (OR, 0.19; CI 95%, 0.05-0.65; P<0.01), correlating with the lack of Ido2−/− murine females with PDAC. A pooled analysis combining the TJUH data set with the TCGA data set (n=200), looking at all PDAC cases yielded similar results, showing a significant absence of females harboring a homozygous R248W (IDO2-deficient) genotype (OR, 0.35; CI 95%, 0.17-0.74; P<0.01).

### C. IDO2 SNPs and Patient PDAC Outcomes

Having obtained genetic evidence implicating IDO2 in pancreatic cancer development, we next examined how IDO2 functional status might influence the responses of PDAC patients to treatment based on the occurrence of the two functionally disruptive SNPs within the coding region of the human IDO2 gene (16). For the purpose of this study, two independent data sets were analyzed, a publicly available data set from The Cancer Genome Atlas Research Network (TCGA-PAAD) and an internally generated data set collected under an IRB-approved study at the Thomas Jefferson University Hospital in Philadelphia (TJUH; refs. 14, 30). Distributions of both IDO2 coding region SNPs rs4503083 (Y359X) and rs10109853 (R248W) in the TCGA-PAAD set were within the normal range of predicted Hardy-Weinberg equilibrium frequencies (_{χ}2 test, P=NS), consistent with previously published data (14). In the TJUH patient data set, a statistically significant 2-fold increase in prevalence of the Y359X homozygous genotype was noted compared with the predicted Hardy-Weinberg frequencies (P<0.05), with additional analysis using Fisher exact comparison supporting some deviation from equilibrium (P<0.05). However, in a second comparison with the 1000 Genome CEU data set (Utah residents with Northern and Western European ancestry) as a normal control, we observed no significant deviation in distribution (30).

D. The TCGA Data Set Includes Histologic Information on NLR for Many of the Resected Tumors, which is Categorized as a Basic Indicator of a Protumorigenic Inflammatory State

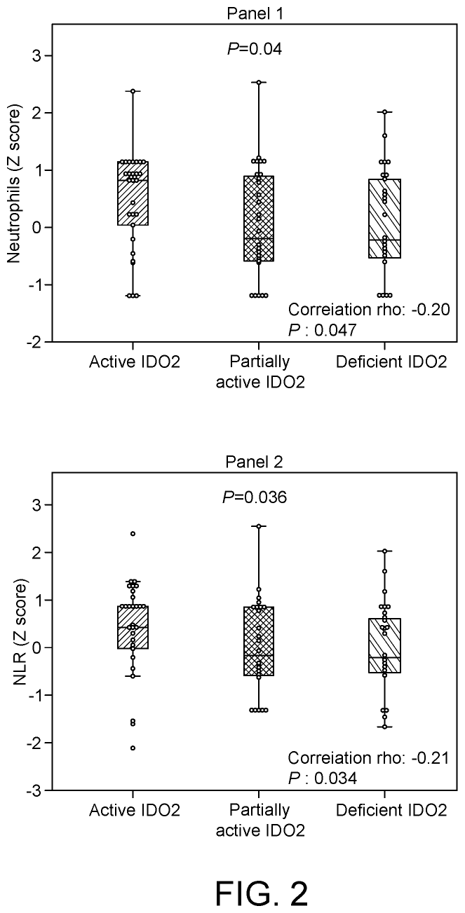

As IDO2 is implicated in immune regulation, the association of IDO2 SNP status with NLR status was interrogated. Pooled analysis of histologic immune data from the TCGA cohort and slides collected from the TJUH cohort (N=99) showed that IDO2-deficient genetic status significantly correlated with decreased neutrophil infiltration and improved (lower) NLR scores (P=0.047 and P=0.034, respectively; FIG. 2). RNA expression data available from the TCGA cohort (N=123) further corroborate these findings. IDO2 genetic status correlated with decreases in the imputed neutrophil to overall-lymphocytes ratio, neutrophil to T-cell ratio, and neutrophil to B-cell ratio, and an increase in expression of cytotoxic lymphocytes (P=0.018, P=0.037, P=0.082, and P=0.018, respectively). These results suggest that the loss of IDO2 function is associated with an improved immune signature.

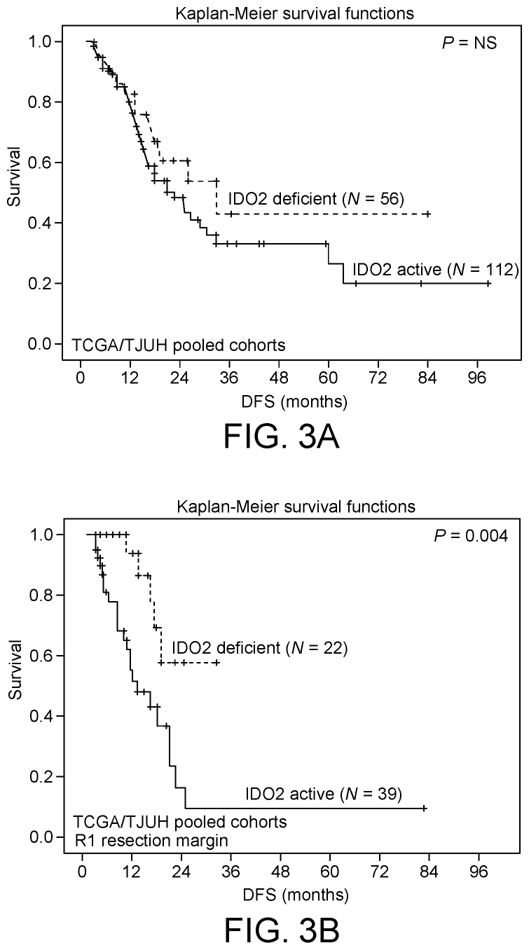

Given the genetic and histologic indications of IDO2 involvement in PDAC development, we explored whether an association could also be drawn between IDO2 genetic status and patient treatment outcomes (31). Due to the high censorship rate (71%) of the DFS data in the TCGA-PAAD cohort, we pooled these data with our TJUH cohort to perform survival analyses. In the overall PDAC patient pool, Kaplan-Meier analysis suggested a trend toward increased DFS in patients with IDO2-deficient status that did not reach a level of significance (median survival 20.3±3.5 vs. 32.4±9.9 months, FIG. 3A). This observation suggested the possibility of a wider differential among a specific subgroup of patients that is masked in the overall patient pool. Indeed, a subanalysis of microscopic tumor involvement (R1 resection margin cases, N=61) revealed that IDO2-deficient status was significantly associated with a favorable prognosis (FIG. 3B, P=0.004) with the IDO2-deficient group not reaching its median survival in 32 months of follow-up. No significant association was seen in related subanalyses based on nodal metastasis, tumor grade or tumor size (≤3 cm or >3 cm) in this cohort (data shown in S3 of ref 51), however, a similar favorable trend was observed in IDO2-deficient patients with large tumors (Size>3 cm, P=0.099). A multivariate Cox regression identified only resection margin status as a statistically significant independent risk factor (HR, 1.81; CI 95%, 1.10-2.97; P=0.02) for DFS. IDO2-deficient status also showed a strong trend toward positive long-term DFS (HR, 0.64; CI 95%, 0.37-1.10; P=0.10). Tumor size, nodal involvement, and tumor grade were not found to be significant. These observations encouraged further analysis of whether specific treatment regimens received by patients with positive R1 resection margins might be influenced by IDO2 genetic status. IDO2-deficient genotype correlates with improved survival of PDAC patients who received adjuvant radiotherapy

In considering the possible mechanisms through which IDO2 status modifies survival in patients with positive resection margins, we investigated the use of adjuvant radiotherapy as a potential mediating factor (32). Reinforcing this exploratory logic was the finding of an association between IDO2-deficient status and reduced NLR, the latter of which has been identified by several studies as a positive prognostic factor for patients receiving radiotherapy for various cancers including PDAC (33). Thus, an aggregate evaluation of the patient data suggested the possibility that IDO2 status might be of relevance to patients who had received adjuvant radiotherapy during their course of treatment.

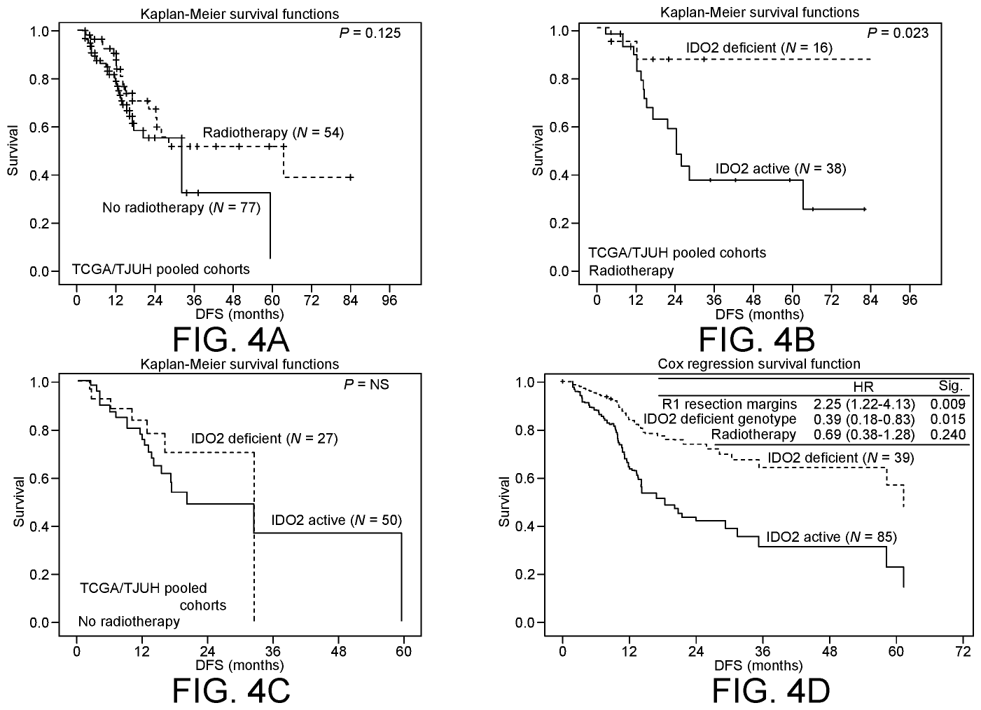

A total of 54 PDAC resection cases with DFS≥2 months and including documentation of administered radiotherapy were available for evaluation in the pooled TCGA-PAAD and TJUH cohorts, together with a corresponding pooled cohort of 77 patients who did not receive radiotherapy. In comparing these two groups, there was no demonstrable improvement in DFS attributable to adjuvant radiotherapy (FIG. 4A), consistent with other studies that have reported a lack of benefit. However, inclusion of the IDO2 status in the survival analysis of the radiotherapy treatment cohort revealed a significant association of the IDO2-deficient genotype with mean DFS almost doubling in duration mean survival (39.0±6.3 vs. 74.1±6.4 months, FIG. 4B, P=0.023) and over 50% survival during the follow-up time (median survival was not reached). Importantly, IDO2 SNP-based stratification of the cohort that did not receive radiotherapy showed no significant evidence of a DFS benefit associated with IDO2-deficient status (FIG. 4C). Elaborating further on the association of IDO2 host genetic status with radiotherapeutic impact, a Cox multivariate hazard analysis of DFS defined IDO2-deficient genotype to be an independent positive factor (HR, 0.39; 95% CI; 0.18-0.83; P=0.015; FIG. 4C). In summary, we conclude from this retrospective analysis that IDO2 host genotype appears to be a significant predictor of DFS in PDAC patients who received adjuvant radiotherapy as part of their care.

Our results provide clear evidence linking IDO2 function to PDAC pathophysiology and therapeutic response. Locoregional degradation of tryptophan and accumulation of kynurenine catabolites have been broadly implicated in supporting cancer-promoting inflammation and immune escape (2, 3, 34). Unlike the IDO1 enzyme that has been the primary focus of attention, IDO2 is less widely expressed in human tumors but has been reported in gastric, colon, and renal cancers (35) as well as in PDAC where it appears to be widely overexpressed (14). Our genetic data from mice support the notion that IDO2 can play a contributory role in PDAC tumorigenesis. In contrast, in the case of familial PDAC, we have recently published evidence of increased risk being associated with an IDO2-deficient genotype status although the trend for sporadic PDAC went in the opposite direction (30). A possible explanation might include a double-edged model in which tumor initiation and tumor progression are located on either side of the IDO2 functionality spectrum. However, addressing the observed discrepancies between the mouse KC model, sporadic PDAC, and familial PDAC awaits detailed cellular and molecular interrogation of IDO2s various effects at the tumor cell, the microenvironment, and systematic immune system levels.

As a metabolic modifier of inflammation, IDO2 is likely to exert complex effects. However, it is notable that preclinical studies of the more extensively studied IDO1 enzyme have clearly established its role in mediating tumoral resistance to DNA-damaging chemotherapies and ionizing radiation which are immunogenic in nature (6, 7). Correspondingly, a recent clinical study of non-small cell lung cancer patients by Wang and colleagues reported that activity levels ascribed to IDO1 corresponded with responsiveness to radiotherapy (36). Specifically, their results showed significant correlations between lower kynurenine/tryptophan ratios assessed pre- and postradiotherapy and prolonged OS. Although no direct determination of the specific enzyme responsible for catabolizing tryptophan to kynurenine in these patients was provided in this study, prior evidence suggests that IDO2 is not likely to be directly responsible for this level of activity as it is enzymatically less active than either IDO1 or TDO and its loss has not been found to affect systemic kynurenine/tryptophan levels (37). However, IDO2 has been shown to enable the promotion of regulatory T-cell activation by IDO1 (11). Thus, the possibility that IDO2 may be indirectly affecting the ability of IDO1 to regulate T-cell function highlights the need to consider IDO2 genetic status in situations where IDO1 has been clinically implicated. Additionally, IDO2 has been shown to have a biological role in supporting pathogenic B-cell antibody production in autoimmune settings (9, 10). This finding is particularly noteworthy given that protumorigenic B cells, initially identified with squamous cell carcinomas of the vulva and the head and neck, have also been clinically associated with PDAC (18, 38), and recent preclinical studies have implicated B cells as contributing to PDAC development (18, 38). The striking absence of adenocarcinomas observed in female Ido2−/− KC mice precluded our ability to evaluate intratumoral B cells in these animals relative to their more susceptible counterparts, while the extremely low levels of B cells detected in the pancreas made comparative analysis of B cells the local microenvironment untenable. However, expression of oncogenic Kras in the pancreas was associated with differences in frequencies of other local immune cell populations, which in several instances appeared to be more pronounced in the animals lacking IDO2. Nevertheless, the trend in these data were not sufficiently robust to rule out the possibility of other functional contributions of IDO2 in this setting, perhaps including nonimmune functions, that are yet to be elucidated. Additional studies will be needed to determine whether any of these associations are functionally relevant to the reduced incidence of PDAC associated with IDO2 loss in female mice. Elucidating the basis for PDAC resistance in this preclinical model may have direct translational relevance given the significant absence of female R248W (IDO2-deficient) SNP representation in our analysis of PDAC A major limitation of our study is the low number of PDAC samples evaluated. Still, our results may have important ramifications for PDAC treatment, most notably about use of adjuvant radiotherapy where variable efficacy has been reported in patients. In the adjuvant setting, PDAC therapy has remained little changed for over the past two decades in relying mainly on 5FU or gemcitabine as monotherapies (39-41). Newer combinations such as gemcitabine/capecitabine have shown some promise but they produce higher rates of toxicity, limiting completion of treatment protocols for some patients (42). In the search for ways to improve standard of care, the benefits of adjuvant radiotherapy have been highly debated. Studies assessing the impact of radiotherapy on survival have produced results ranging from increased OS (43-45) to no response or even poorer response (46-48). In our pooled analysis of the TCGA-PAAD and TJUH cohorts, we identified a significant association between a functionally ablated host IDO2 genotype and a favorable response to radiotherapy. Given the significant natural variation in the IDO2 coding SNP genotypes among human populations (16), our findings offer a potentially incisive explanation for the large variability in efficacy reported for adjuvant radiotherapy. Thus, IDO2 genotyping may provide a biomarker to personalize this modality for PDAC patients. Further evaluation of the host IDO2 gene as a predictive biomarker is warranted to confirm and extend its utility in additional patient populations. Finally, our results may also have implications regarding the potential for developing IDO2 inhibitors for use in combination with radiotherapy to treat those PDAC patients harboring IDO2-active alleles. Some support for this general concept is provided by a recent pilot study in which stratifying brain metastasis patients for the IDO2-active genotype distinguished a positive trend in OS following whole brain radiotherapy together with coadministration of low-dose chloroquine, an indirect inhibitor of IDO2 but not IDO1 activity (31). Although most experimental agents in clinical trials at present are selective for IDO1, and no IDO2-specific enzyme inhibitors are yet available, the IDO pathway inhibitor indoximod (D-1MT) has a different mechanism of action that may encompass IDO2 blockade (2, 9, 16, 37, 49). Indeed, D, L-1MT administration has been reported to improve the efficacy of radiotherapy in mouse tumor models (7, 50). Thus, in theory, the IDO2 genotype has the potential to serve as a decisional biomarker for distinguishing between PDAC patients who are more likely to respond to adjuvant radiotherapy alone versus those patients who may benefit from the coordinated blockade of IDO2.

### EXAMPLE 2: GENOTYPING PROTOCOL FOR IDO2

A genotyping protocol is performed by obtaining a biological sample, blood or tissue (e.g., surgically resected pancreatic tissue specimens (normal and tumor tissues) or blood). DNA is extracted from the sample using the DNAeasy Blood and Tissue Kit genomic DNA purification kit (Qiagen Inc.). DNA fragments containing the IDO2-coding region polymorphisms rs4503083 (Exon 11) and rs10109853 (Exon 9) are amplified by PCR using the IDO2 oligonucleotide primers R248W forward and R248W reverse; Y359X forward and Y359X reverse, as described in Witkiewicz A K, et al. Genotyping and expression analysis of IDO2 in human pancreatic cancer: a novel, active target. J Am Coll Surg 2009; 208:781-7). These primers are identified in Table 4.

TABLE 4
Sequencing primers for IDO2
functional polymorphisms.

| | R248W | SEQ | Y359X | SEQ |
|---|---|---|---|---|
| | (rs10109853) | ID NO | (rs4503083) | ID NO |
| Forward | 5′-GAACATTCTATC | 1 | 5′-TCTTGTGCTCC | 3 |
| | CCCCGTTGC-3′ | | CTCCAAAACA-3′ | |
| Reverse | 5′-TTACCTGAGAGT | 2 | 5′-TGGTTTGGCTT | 4 |
| | GGATCCCTAGCA-3′ | | CCCATGCTT-3′ | |

PCR reactions are performed in 25 μL using 100 ng of gDNA, 0.5 μg/μL of Taq polymerase (Affymetrix), 1 μL of 10 μmol/L oligonucleotide primers (forward and reverse), 2.5 μL of 10X PCR buffer (Affymetrix), and 0.5 μL 10 mmol/L dNTP Mix (Affymetrix). PCR reaction products are purified using a commercial PCR purification kit (Qiagen Inc.). Each PCR reaction is examined by gel electrophoresis on a 0.75% DNA agarose gel before Sanger sequencing by a commercial provider (GenScript Inc.) using the DNA oligonucleotide primers mentioned above. Genotyping steps are blinded to clinical data and familial-sporadic patient status. IDO2 genotype in patients is determined by chromatogram (Witkiewicz A K, et al, cited above).

### EXAMPLE 3: EVALUATION OF IMPACT OF GENOTYPE ON THERAPEUTIC EFFICACY IN PDAC PATIENTS

### A. Protocol

The treatment protocol was as described in ClinicalTrials.gov Identifier NCT02077881, which was a Phase I/II trial designed to evaluate the combination of the immunotherapeutic agent indoximod (an IDO inhibitor) and the standard of care chemotherapy gemcitabine plus nab-paclitaxel in subjects with metastatic adenocarcinoma of the pancreas. Participants received oral indoximod (600 mg, 100 mg, or 1200 mg according to their assigned dose cohort) twice daily for 28 days concurrently with IV Nab-paclitaxel 125 mg/m² given intravenously over 30-40 minutes for 3 weeks (days 1, 8 and 15) with 1 week rest, followed by gemcitabine 1000 mg/m² given intravenously over 30 minutes for 3 weeks (days 1, 8 and 15) with 1 week rest. All subjects will receive the standard 28-day gemcitabine plus nab-paclitaxel regimen. Twice daily oral indoximod was administered concurrently in continuous 28 day cycles. Patients continued until they experienced disease progression or significant toxicity.

Genotyping to detect the wild-type IDO2 alleles or a polymorphisms in one or both alleles was performed using a protocol as provided in Example 2.

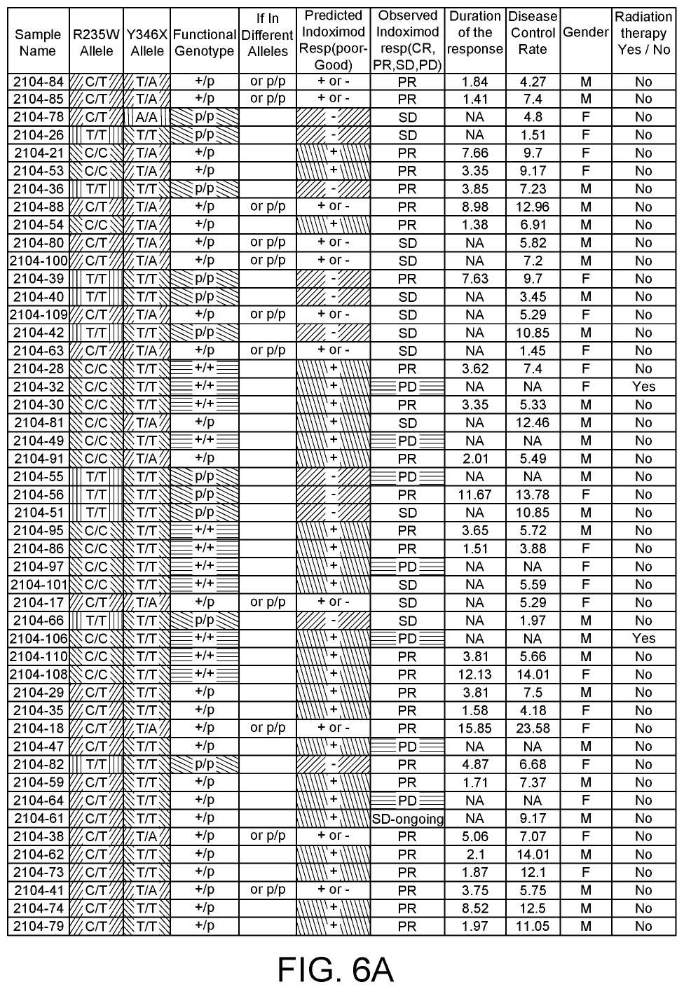

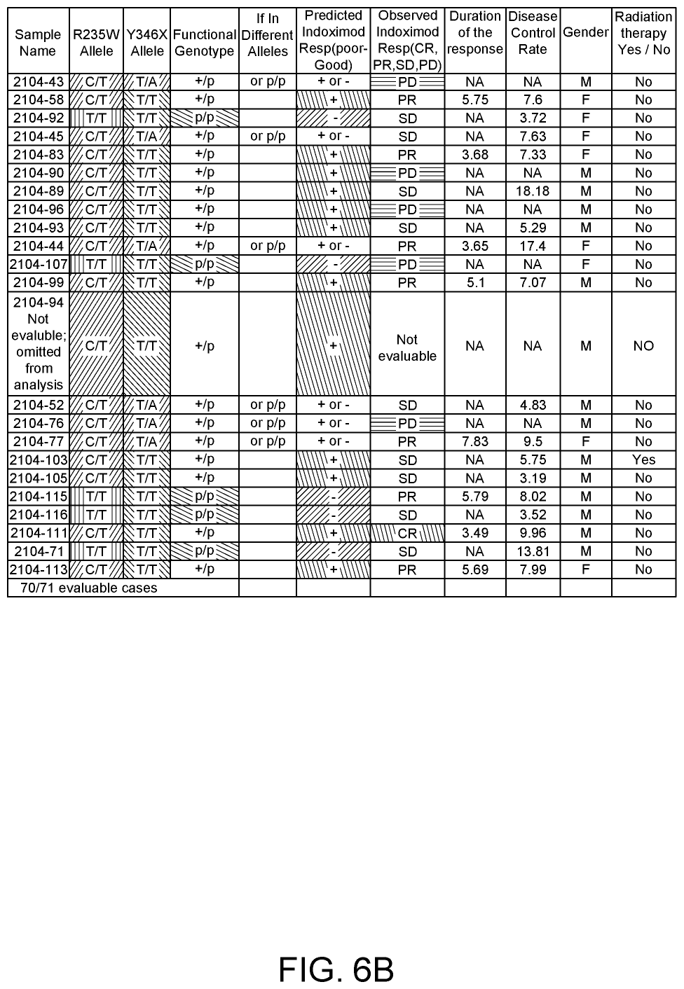

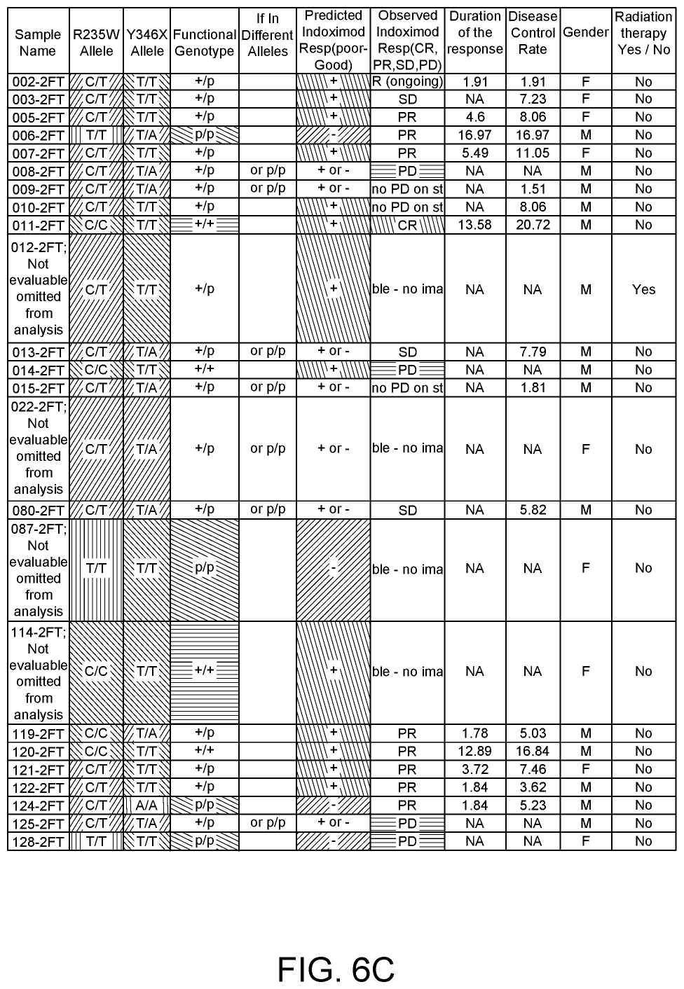

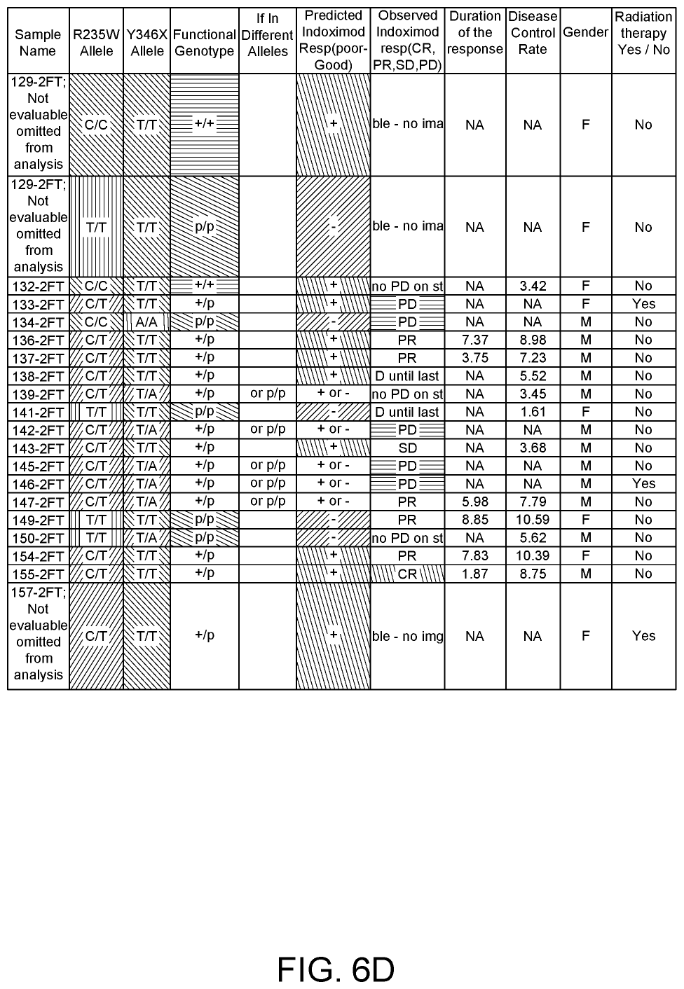

Two-sample tests of independent proportions were performed to compare the percentage of positive responders between two groups. Observed versus expected genotype distributions were compared using one-sample tests of proportions. The significance level was set to 0.05 and all tests were one-sided except for the tests comparing response by sex and the tests comparing observed and expected genotypes. Analyses were performed in Stata/MP 15.1 (StataCorp LP., Texas, USA). See the data in FIG. 6A -6D.

TABLE 5
IDO2 genotype functionality definitions.
A three-tier scale describes possible genotype
combinations of IDO2 functional polymorphisms.

| IDO2 Functionality Grade | R248W Genotype | | Y359X Genotype | |
|---|---|---|---|---|
| Fully Active | R248 | R248 | Y359 | Y359 |
| Predicted Partially | R248 | 248W | Y359 | Y359 |
| Active | R248 | R248 | 359X | Y359 |
| | R248 | 248W | Y359 | 359X |
| Deficient | 248W | 248W | Y359 | Y359 |
| | 248W | 248W | 359X | Y359 |
| | 248W | 248W | 359X | 359X |
| | 248W | R248 | 359X | 359X |
| | R248 | R248 | 359X | 359X |

### B. Overall Cohort Analysis

Excludes (+/p or p/p) uncertain genotype. n=79 (3 CRs, 41 PRs, 21 SDs, 14 PDs)

TABLE 6
Distribution of Responses by Genotype (Row %s Shown)

| Genotype | SD + PD | PR + CR | Total |
|---|---|---|---|
| +/+ | 7 (46.7) | 8 (53.3) | 15 |
| +/p | 14 (34.2) | 27 (65.9) | 41 |
| p/p | 14 (60.9) | 9 (39.1) | 23 |
| Total | 35 | 44 | 79 |

Results: 53.3% of the +/+, 65.9% of the +/p, and 39.1% of the p/p genotypes had a positive (PR+CR) response. There was no significant difference in the percentage of positive responses between the +/+ and p/p genotypes (53.3% vs 39.1%, p=0.195).

There was a significant difference in the percentage of positive responses between the +/p and p/p genotypes (65.9% vs 39.1%, p=0.019). Specifically, those with the p/p genotype had a lower percentage of positive responses.

TABLE 7
Distribution of Responses by Collapsed Genotype (Row %s Shown)

| Genotype | SD + PD | PR + CR | Total |
|---|---|---|---|
| +/+ or +/p | 21 (37.5) | 35 (62.5) | 56 |
| p/p | 14 (60.9) | 9 (39.1) | 23 |
| Total | 35 | 44 | 79 |

Results:

62.5% of the (+/+ or +/p) and 39.1% of the p/p genotypes had a positive response.

There was a significant difference in the percentage of positive responses between the (+/+ or +/p) and p/p genotypes (62.5% vs 39.1%, p=0.029). Specifically, the (+/+ or +/p) genotype had a significantly higher percentage of positive responders.

### B. Analysis by Sex—Males Only

Excludes (+/p or p/p) uncertain genotype. n=47 males (3 CRs, 21 PRs 15 SDs, 8 PDs)

TABLE 8
Distribution of Responses by Genotype-Males Only (Row %s Shown)

| Genotype | SD + PD | PR + CR | Total |
|---|---|---|---|
| +/+ | 3 (37.5) | 5 (62.5) | 8 |
| +/p | 11 (42.3) | 15 (57.7) | 26 |
| p/p | 9 (69.2) | 4 (30.8) | 13 |
| Total | 23 | 24 | 47 |

Results: 62.5% of the +/+, 57.7% of the +/p, and 30.8% of the p/p genotypes had a positive response. There was not a significant difference in the percentage of positive responders between the +/+ and p/p genotypes (62.5% vs 30.8%, p=0.077) and the +/p and p/p genotypes (57.7% vs 30.8%, p=0.056).

TABLE 9
Distribution of Responses by Collapsed
Genotype-Males Only (Row %s Shown)

| Genotype | SD + PD | PR + CR | Total |
|---|---|---|---|
| +/+ or +/p | 14 (41.2) | 20 (58.8) | 34 |
| p/p | 9 (69.2) | 4 (30.8) | 13 |
| Total | 23 | 24 | 47 |

Results: 58.8% of the (+/+ or +/p) and 30.8% of the p/p genotypes had a positive response. There was a significant difference in the percentage of positive responders between the (+/+ or +/p) and p/p genotypes (58.8% vs 30.8%, p=0.043). Specifically, the (+/+ or +/p) genotype had a significantly higher percentage of positive responders.

### C. Analysis by Sex—Females Only

Excludes (+/p or p/p) uncertain genotype. n=32 females (20 PRs, 6 SDs, 6 PDs

TABLE 10
Distribution of Responses by Genotype-Females Only (Row %s Shown)

| Genotype | SD + PD | PR + CR | Total |
|---|---|---|---|
| +/+ | 4 (57.1) | 3 (42.9) | 7 |
| +/p | 3 (20.0) | 12 (80.0) | 15 |
| p/p | 5 (50.0) | 5 (50.0) | 10 |
| Total | 12 | 20 | 32 |

Results: 42.9% of the +/+, 80.0% of the +/p, and 50.0% of the p/p genotypes had a positive response. There was no significant difference in the percentage of positive responders between the +/+ and p/p genotypes (42.9% vs 50.0%, p=0.614). There was no significant difference in the percentage of positive responders between the +/p and p/p genotypes (80.0% vs 50.0%, p=0.058). Of note, there was a significant difference in the percentage of positive responders between the +/+ and +/p genotypes (42.9% vs 80.0%, p=0.041). Specifically, the +/p genotype had a significantly higher percentage of positive responders.

TABLE 11
Distribution of Responses by Collapsed
Genotype-Females Only (Row %s Shown)

| Genotype | SD + PD | PR + CR | Total |
|---|---|---|---|
| +/+ or +/p | 7 (31.8) | 15 (68.2) | 22 |
| p/p | 5 (50.0) | 5 (50.0) | 10 |
| Total | 12 | 20 | 32 |

Results: 68.2% of the (+/+ or +/p) and 50.0% of the p/p genotypes had a positive response. There was no significant difference in the percentage of positive responders between the (+/+ or +/p) and p/p genotypes (68.2% vs 50.0%, p=0.162)

### D. Comparison by Sex—No Exclusions

n=107; 67 males (3 CRs, 26 PRs, 23 SDs, 15 PDs), 40 females (0 CRs, 24 PRs, 10 SDs, 6 PDs)

TABLE 12
Distribution of Responses by Sex (Row %s Shown)

| Sex | SD + PD | PR + CR | Total |
|---|---|---|---|
| Males | 38 (56.7) | 29 (43.3) | 67 |
| Females | 16 (40.0) | 24 (60.0) | 40 |
| Total | 54 | 53 | 107 |

Results: In the whole cohort, 43.3% of males and 60.0% of females had a positive response. There was no significant difference between the percentage of positive responders between males and females (43.3% vs 60.0%, p=0.094).

E. Comparison by Sex—Excludes (+/p or p/p) Uncertain Genotype

Excludes (+/p or p/p) uncertain genotype. n=79; 47 males (3 CRs, 21 PRs, 15 SDs, 8 PDs), 32 females (0 CRs, 20 PRs, 6 SDs, 6 PDs)

TABLE 13
Distribution of Responses by Sex (Row %s Shown)

| Sex | SD + PD | PR + CR | Total |
|---|---|---|---|
| Males | 23 (48.9) | 24 (51.1) | 47 |
| Females | 12 (37.5) | 20 (62.5) | 32 |
| Total | 35 | 44 | 79 |

Results: In the cohort which excludes the (+/p or p/p) genotype, 51.1% of the males and 62.5% of the females had a positive response. There was no significant difference in the percentage of positive responders between males and females (51.1% vs 62.5%, p=0.315).).

### F. Comparison by Genotype—Both Sexes—No Exclusions

Includes (+/p or p/p) uncertain genotype. n=107 (3 CRs, 50 PRs, 33 SDs, 21 PDs)

TABLE 14
Distribution of Responses by Genotype-
Both Sexes-(Row %s Shown)-Includes (+/p or p/p)

| Genotype | SD + PD | PR + CR | Total |
|---|---|---|---|
| +/+ or +/p | 21 (37.5) | 35 (62.5) | 56 |
| (+/p or p/p) or p/p | 33 (64.7) | 18 (35.3) | 51 |
| Total | 54 | 53 | 107 |

Results: 62.5% of the (+/+ or +/p) and 35.3% of the (+/p or p/p) or p/p genotypes had a positive response. There was a significant difference in the percentage of positive responders between the (+/+ or +/p) and the (+/p or p/p) or p/p genotypes (62.5% vs 35.3%, p=0.005).

### G. Comparison by Genotype—Both Sexes—Excluding Uncertain Genotype

Excludes (+/p or p/p) uncertain genotype. n=79 (3 CRs, 41 PRs, 21 SDs, 14 PDs)

TABLE 15
Distribution of Responses by Genotype-
Both Sexes-(Row %s Shown)-Excludes (+/p or p/p)

| Genotype | SD + PD | PR + CR | Total |
|---|---|---|---|
| +/+ or +/p | 21 (37.5) | 35 (62.5) | 56 |
| p/p | 14 (60.9) | 9 (39.1) | 23 |
| Total | 35 | 44 | 79 |

Results: 62.5% of the (+/+ or +/p) and 39.1% of the p/p genotypes had a positive response. There was a significant difference in the percentage of positive responses between the (+/+ or +/p) and p/p genotypes (62.5% vs 39.1%, p=0.029). Specifically, the (+/+ or +/p) genotype had a significantly higher percentage of positive responders.

### H. Comparison by Genotype—Males Only—No Exclusions

Includes (+/p or p/p) uncertain genotype; n=67 (3 CRs, 26 PRs, 23 SDs, 15 PDs)

TABLE 16
Distribution of Responses by Genotype - Males
Only - (Row %s Shown) - Includes (+/p or p/p)

| Genotype | SD + PD | PR + CR | Total |
|---|---|---|---|
| +/+ or +/p | p14 (41.2) | 20 (58.8) | 34 |
| (+/p or p/p) or p/p | p24 (72.7) | 9 (27.3) | 33 |
| Total | 38 | 29 | 67 |

Results: 58.8% of the (+/+ or +/p) and 27.3% of the (+/p or p/p) or p/p genotypes had a positive response. There was a significant difference in the percentage of positive responders between the (+/+ or +/p) and the (+/p or p/p) or p/p genotypes (58.8% vs 27.3%, p=0.005)

### I. Comparison by Genotype—Males Only—Excluding Uncertain Genotype

Excludes (+/p or p/p) uncertain genotype
n=47 (3 CRs, 21 PRs, 15 SDs, 8 PDs)

TABLE 17
Distribution of Responses by Genotype - Males
Only - (Row %s Shown) - Excludes (+/p or p/p)

| Genotype | SD + PD | PR + CR | Total |
|---|---|---|---|
| +/+ or +/p | 14 (41.2) | 20 (58.8) | 34 |
| p/p | 9 (69.2) | 4 (30.8) | 13 |
| Total | 23 | 24 | 47 |

Results: 58.8% of the (+/+ or +/p) and 30.8% of the p/p genotypes had a positive response. There was a significant difference in the percentage of positive responders between the (+/+ or +/p) and p/p genotypes (58.8% vs 30.8%, p=0.043). Specifically, the (+/+ or +/p) genotype had a significantly higher percentage of positive responders.

### J. Analysis by Expected Genotype—Both Sexes

TABLE 18
Expected Genotype Distribution vs Observed Distribution (Overall Cohort
(n = 107) and Positive Responders Only (n = 53))

| | | Observed Genotype | | Observed Genotype | |
|---|---|---|---|---|---|
| | | Distribution in | | Distribution in | |
| | Expected Genotype | Pancreas Cancer | | Positive Responders | |
| | Distribution | Patient Cohort (%) | | to Therapy | |
| | (Racially Weighted | ALL patients | | (CR + PR) (%) | |
| Genotype | to Cohort) | (N = 107) | p-value | (N = 53) | p-value |
| +/+ | 12.9% | 14.0% (15/107) | 0.730 | 15.1% (8/53) | 0.634 |
| +/p | 46.5% | 38.3% (41/107) | 0.090 | 50.9% (27/53) | 0.517 |
| p/p | 40.6% | 21.5% (23/107) | <0.001 | 17.0% (9/53) | <0.001 |
| +/+ or +/p | 59.40% | 52.3% (56/107) | 0.137 | 66.0% (35/53) | 0.325 |

Results: In the full cohort (n=107), 14% of patients were +/+, 38.3% were +/p, and 21.5% were p/p. The observed percentage of p/p patients was significantly different than what was expected (21.5% vs 40.6%, respectively; p=<0.001). Specifically, the observed percentage of p/p patients was lower than what was expected. None of the other genotypes were significantly different than what was expected.

In the positive responders (n=53), 15.1% of patients were +/+, 50.9% were +/p, and 17.0% were p/p. The observed percentage of p/p patients was significantly different than what was expected (17.0% vs 40.6%, respectively; p=<0.001). Specifically, the observed percentage of p/p patients was lower than what was expected. None of the other genotypes were significantly different than what was expected.

TABLE 19
Expected Genotype Distribution vs Observed Distribution - Excluding Uncertain
Genotypes (Overall Cohort (n = 79) and Positive Responders Only (n = 44))

| | | | | Observed Genotype | |
|---|---|---|---|---|---|
| | | Observed Genotype | | Distribution in | |
| | | Distribution in | | Positive Responders | |
| | | Pancreas Cancer | | to Therapy | |
| | Expected Genotype | Patient Cohort (%) | | (CR + PR) (%) | |
| | Distribution | OMIT uncertain | | OMIT uncertain | |
| | (Racially Weighted | genotype patients | | genotype patients | |
| Genotype | to Cohort) | (N = 79) | p-value | (N = 44) | p-value |
| +/+ | 12.9% | 19.0% (15/79) | 0.107 | 18.2% (8/44) | 0.296 |
| +/p | 46.5% | 51.9% (41/79) | 0.336 | 61.4% (27/44) | 0.048 |
| p/p | 40.6% | 29.1% (23/79) | 0.038 | 20.5% (9/44) | 0.007 |

Results: In the full cohort (n=79 after omitting uncertain genotype group), 19.0% of patients were +/+, 51.9% were +/p, and 29.1% were p/p. The observed percentage of the p/p genotype was significantly different than expected (29.1% vs 40.6%, respectively; p=0.038). Specifically, the percentage of observed p/p patients was significantly lower than what was expected. None of the other genotypes were significantly different than what was expected.

In the cohort of positive responders (n=44 after omitting uncertain genotype group), 18.2% of the patients were +/+, 61.4% were +/p, and 20.5% were p/p. The observed percentage of the p/p genotype was significantly different than expected (20.5% vs 40.6%, respectively; p=0.007). Specifically, the observed percentage of the p/p genotype was significantly lower than what was expected. Additionally, the observed percentage of the +/p genotype was significantly different than expected (61.4% vs 46.5%, respectively; p=0.048). Specifically, the percentage observed was significantly higher than expected.

TABLE 20
Expected Genotype Distribution vs Observed Distribution (Overall Male Cohort
(n = 67) and Positive Male Responders Only (n = 29))

| | | | | Observed Genotype | |
|---|---|---|---|---|---|
| | | Observed Genotype | | Distribution in | |
| | | Distribution in | | Positive Male | |
| | Expected Genotype | Pancreas Cancer | | Responders | |
| | Distribution | Patient Cohort (%) | | to Therapy | |
| | (Racially Weighted | All Male Patients | | (CR + PR) (%) | |
| Genotype | to Cohort) | (N = 67) | p-value | (N = 29) | p-value |
| +/+ | 12.9% | 11.9% (8/67) | 0.815 | 17.2% (5/29) | 0.486 |
| +/p | 46.5% | 38.8% (26/67) | 0.207 | 51.7% (15/29) | 0.573 |
| p/p | 40.6% | 19.4% (13/67) | <0.001 | 13.8% (4/29) | 0.003 |
| +/+ or +/p | 59.40% | 50.8% (34/67) | 0.149 | 69.0% (20/29) | 0.294 |

Results: In the overall male cohort (n=67), 11.9% of the patients were +/+, 38.8% were +/p, and 19.4% were p/p. The observed percentage of the p/p genotype was significantly different than expected (19.4% vs 40.6%, respectively; p=<0.001). Specifically, the observed percentage of the p/p genotype was significantly lower than what was expected. None of the other genotypes were significantly different than what was expected. In the cohort of male positive responders (n=29), 17.2% of the patients were +/+, 51.7% were +/p, and 13.8% were p/p. The observed percentage of the p/p genotype was significantly different than expected (13.8% vs 40.6%, respectively; p=0.003). Specifically, the observed percentage of the p/p genotype was significantly lower than expected. None of the other genotypes were significantly different than what was expected.

TABLE 21
Expected Genotype Distribution vs Observed Distribution - Excluding Uncertain
Genotypes (Male Cohort (n = 47) and Positive Male Responders Only (n = 24))

| | | | | Observed Genotype | |
|---|---|---|---|---|---|
| | | Observed Genotype | | Distribution in | |
| | | Distribution in | | Positive Male | |
| | | Male Pancreas | | Responders | |
| | | Cancer Patient | | to Therapy | |
| | Expected Genotype | Cohort (%) | | (CR + PR) (%) | |
| | Distribution | OMIT uncertain | | OMIT uncertain | |
| | (Racially Weighted | genotype patients | | genotype patients | |
| Genotype | to Cohort) | (N = 47) | p-value | (N = 24) | p-value |
| +/+ | 12.9% | 17.0% (8/47) | 0.399 | 20.8% (5/24) | 0.246 |
| +/p | 46.5% | 55.3% (26/47) | 0.225 | 62.5% (15/24) | 0.116 |
| p/p | 40.6% | 27.7% (13/47) | 0.071 | 16.7% (4/24) | 0.017 |

Results: In the overall male cohort (n=47 after omitting uncertain genotypes), 17.0% of the patients were +/+, 55.3% were +/p, and 27.7% were p/p. Within any genotype, there were no significant differences between what was observed and what was expected. In the cohort of positive male responders (n=24 after omitting uncertain genotypes), 20.8% of patients were +/+, 62.5% were +/p, and 16.7% were p/p. The observed percentage of the p/p genotype was significantly different than expected (16.7% vs 40.6%, respectively; p=0.017). Specifically, the observed percentage was significantly lower than what was expected.

### L. Analysis by Expected Genotype — Females Only

TABLE 22
Expected Genotype Distribution vs Observed Distribution (Overall Female Cohort
(n = 40) and Positive Female Responders Only (n = 24))

| | | Observed Genotype | | Observed Genotype | |
|---|---|---|---|---|---|
| | | Distribution in | | Distribution in | |
| | | Female Pancreas | | Positive Female | |
| | Expected Genotype | Cancer Patient | | Responders | |
| | Distribution | Cohort (%) | | to Therapy | |
| | (Racially Weighted | All Female patients | | (CR + PR) | |
| Genotype | to Cohort) | (N = 40) | p-value | (N = 24) | p-value |
| +/+ | 12.9% | 17.5% (7/40) | 0.385 | 12.5% (3/24) | 0.953 |
| +/p | 46.5% | 37.5% (15/40) | 0.254 | 50.0% (12/24) | 0.731 |
| p/p | 40.6% | 25.0% (10/40) | 0.045 | 20.8% (5/24) | 0.049 |
| +/+ or +/p | 59.40% | 55.0% (22/40) | 0.571 | 62.5% (15/24) | 0.757 |

Results: In the overall female cohort (n=40), 17.5% of the patients were +/+, 37.5% were +/p, and 25.0% were p/p. The observed percentage of the p/p genotype was significantly different than expected (25.0% vs 40.6%, respectively; p=0.045). Specifically, the observed percentage was significantly lower than expected. For the other two genotypes, there were no significant differences between what was observed and what was expected. In the cohort of positive female responders (n=24), 12.5% of the patients were +/+, 50.0% were +/p, and 20.8% were p/p. The observed percentage of the p/p genotype was significantly different than expected (20.8% vs 40.6%, respectively; p=0.049). Specifically, the observed percentage was significantly lower than expected. For the other two genotypes, there were no significant differences between what was observed and what was expected.

TABLE 23
Expected Genotype Distribution vs Observed Distribution - Excluding Uncertain Genotype
(Overall Female Cohort (n = 32) and Positive Female Responders Only (n = 20))

| | | | | Observed Genotype | |
|---|---|---|---|---|---|
| | | Observed Genotype | | Distribution in | |
| | | Distribution in | | Positive Female | |
| | | Female Pancreas | | Responders | |
| | | Cancer Patient | | to Therapy | |
| | Expected Genotype | Cohort (%) | | (CR + PR) (%) | |
| | Distribution | OMIT uncertain | | OMIT uncertain | |
| | (Racially Weighted | genotype patients | | genotype patients | |
| Genotype | to Cohort) | (N = 32) | p-value | (N = 20) | p-value |
| +/+ | 12.9% | 21.9% (7/32) | 0.130 | 15.0% (3/20) | 0.779 |
| +/p | 46.5% | 46.9% (15/32) | 0.966 | 60.0% (12/20) | 0.226 |
| p/p | 40.6% | 31.3% (10/32) | 0.282 | 25.0% (5/20) | 0.155 |

Results: In the overall female cohort (n=32 after omitting uncertain genotypes), 21.9% of patients were +/+, 46.9% were +/p, and 31.3% were p/p. For any of the genotypes, there were no significant differences between what was observed and what was expected. In the cohort of positive female responders (n=20 after omitting uncertain genotypes), 15.0% of patients were +/+, 60.0% were +/p, and 25.0% were p/p. For any genotypes, there were no significant differences between what was observed and what was expected.

### EXAMPLE 4: UPDATED ANALYSIS OF PDAC STUDY

This study was conducted to establish the hypotheses that IDO2 inactive genotypes are associated with improved survival for resectable PDAC patients; and that metastasis is the main prognostic factor of mortality from PDAC. The inventors considered that if IDO2 inactive genotypes confer a survival advantage, there will be an unexpected paucity of inactive genotypes amongst metastatic PDAC patients.

The First cohort was a group of 61 metastatic PDA patients and CEU Cohort (1000 genome) was a normal population (n=100). Both cohorts were made up of persons of white European ancestry. Both SNPs were examined for the cohorts including co-occurrence. IDO2 activity genotypes were extracted (including allele-specific SNPs for double-heterozygous). Table 24 shows the genotype distribution comparison; and shows that the First cohort (metastatic PDA subjects) contains a significant decrease in the homozygous SNPs.

TABLE 24

| | First Cohort (white; | | | | | |
|---|---|---|---|---|---|---|
| | metastatic PDA) | | CEU Cohort (white) | | | |
| | n = 61 | | n = 99 | | P Value | |
| Genotype | RW | YX | RW | YX | RW | YX |
| WT/WT | 23 (38%) | 42 (69%) | 26 (26%) | 54 (54%) | 0.000867 | 0.12 |
| Heterozygous +/p | 35 (57% | 18 (29%) | 44 (44%) | 38 (38%) | | |
| Homozygous | 3 (5%) | 1 (2%) | 29 (30%) | 7 (7%) | | |
| p/p | | | | | | |
| Total | 61 (100%) | 61( 100%) | 99 (100%) | 99 (100%) | | |

The IDO2 activity genotype distribution among the First cohort and the control CEU cohort is shown in Tables 25-27. Tables 25-27 demonstrate that the inactive IDO2 genotypes are 5− less abundant in the metastatic PDA cohort compared to the normal population control.

TABLE 25

| | First Cohort | CEU Cohort | |
|---|---|---|---|
| | (white; metastatic) | (white; normal) | |
| IDO2 Activity | n = 61 | n = 99 | P value |
| Active/Active | 23 (38%) | 11 (11%) | 0.000004 |
| Active/Partial | 19 (31%) | 40 40%) | |
| Partial/Partial | 15 (25%) | 14 (14%) | |
| Inactive/Any | 4 (6%) | 34 (35%) | |
| Total | 61 (100%) | 99 (100)% | |

TABLE 26

| | First Cohort | CEU Cohort | |
|---|---|---|---|
| | (white; metastatic) | (white; normal) | |
| IDO2 Activity | n = 61 | n = 99 | P value |
| Probably Active | 42 (38%) | 51 (52%) | 0.000261 |
| Partial/Partial | 15 (25%) | 14 (14%) | |
| Inactive/Any | 4 (6%) | 34 (35%) | |
| Total | 61 (100%) | 99 (100)% | |

TABLE 27

| | First Cohort | CEU Cohort | | |
|---|---|---|---|---|
| | (white; metastatic PDA) | (white; normal) | | |
| Summary | n = 61 | n = 99 | P value | OR |
| WT/heterozygous | 57 (93%) | 65 (66%) | 0.000099 | 7.4538 |
| Inactive | 4 (7%) | 34 (34%) | | |
| Total | 61 (100%) | 99 (100%) | | |

The following Table 28 presents an ‘in silico’ analysis of a public TCGA dataset for a cohort of PDAC patients (not a trial but just a genomic comparison to progression outcomes). Briefly, this data supports that PDAC patients with inactive IDO2 may be less likely to progress to the most advanced stages of disease. This analysis illustrates the ability of the assay to predict the survival outcome of an early-stage patient who has been surgically resected, based on their IDO2 gene status. TCGA data was collected from 147 patients who were Stage I/II PDAC and 14 patients who had advanced/metastatic PDAC as shown in Table 28 below also suggests a paucity in inactive IDO2 genotypes, especially in advanced PDAC (compared to early stage PDAC and to normal population cohort).

TABLE 28

| | CEU Cohort | | TCGA | | TCGA |
|---|---|---|---|---|---|
| | (white; normal) | | Stage I/II | | Advanced/Metastatic |
| IDO2 Activity | n = 99 | P Value | PDAC | P Value | PDAC |
| Probably Active | 51 (52%) | | 99 (67%) | 0.005 | 7 (50%) |
| Partial/Partial | 14 (14%) | | 22 (15%) | | 7 (50%) |
| Inactive/Any | 34 (35%) | 0.01→ | 26 (18%) | 0.0008→ | 0 (0%) |
| Total | 99 (100)% | | 147 (100%) | | 14 (100%) |

### EXAMPLE 5: EVALUATION OF IMPACT OF GENOTYPE ON THERAPEUTIC EFFICACY IN MELANOMA PATIENTS

In related study, evidence indicates that IDO2, a positive modifier in inflammatory disease models, is frequently upregulated in melanoma. As with PDAC above, an inactive IDO2 genetic configuration is associated with extended overall survival if adjuvant radiotherapy, including treatment with an IDO inhibitor, indoximod, was administered.

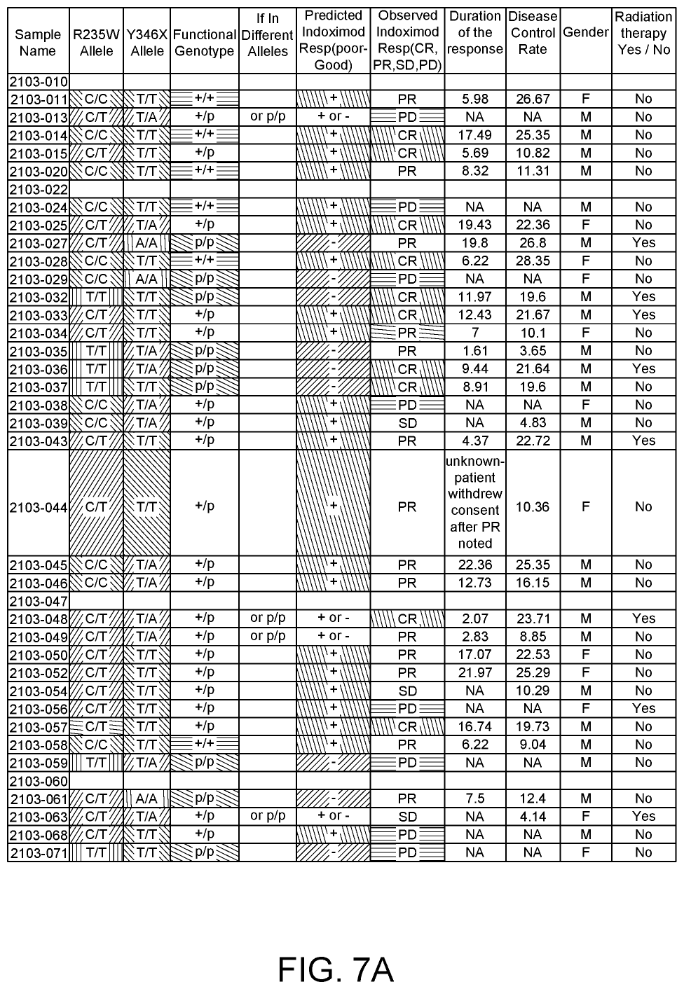

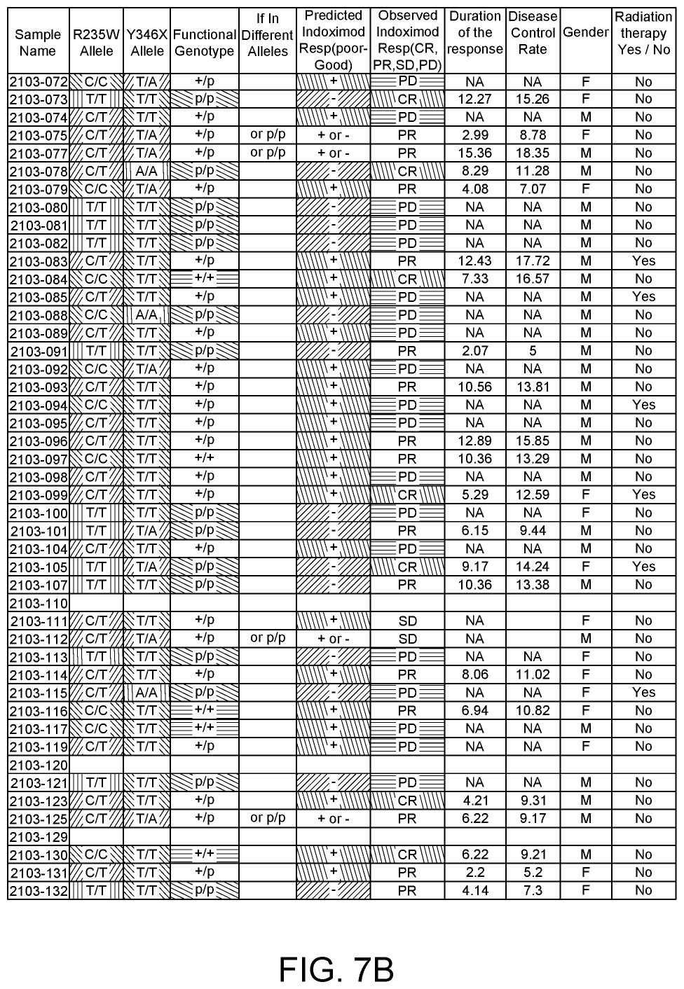

In the melanoma cohort, 84 patients were genotyped with 6 patients returning uncertain results. The remaining 78 patients, including 52 males and 26 females, were treated and analyzed in a similar way to the PDAC patients of Example 3 above. The results of the 46 patients, including 31 males and 15 females, in this cohort that responded positively to IDO and/or other indicated therapies were also analyzed. See the data reported in FIGS. 7A and 7B.

TABLE 29
Expected Genotype Distribution vs Observed Distribution
in Melanoma Patients (Overall Cohort (n =
78) and Positive Responders Only (n = 46))

| | Expected Genotype | Cohort Stratified | Positive Responders |
|---|---|---|---|
| | Distribution | by Genotype(%) | Stratified by |
| | (Racially Weighted | ALL patients | Genotype (%) |
| Genotype | to Cohort) | (N = 78) | (N = 46) |
| +/+ | 10.3% | 15.4% (12/78) | 19.6% (9/46) |
| +/p | 46.3% | 43.6% (34/78) | 41.3% (19/46) |
| +/+ or +/p | 56.6% | 59.0% (46/78) | 60.9% (28/46) |
| p/p | 43.3% | 30.8% (24/78) | 28.3% (13/46) |

Results: In the full cohort (n=78), 15.4% of patients were +/+, 43.6% were +/p, and 30.8% were p/p. The observed percentage of p/p patients lower than what was expected (30.8% vs 43.3%, respectively). The observed percentage of +/+ patients was higher than what was expected (15.4% vs 10.3% respectively).

In the positive responders (n=46), 19.6% of patients were +/+, 41.3% were +/p, and 28.3% were p/p. The observed percentage of +/+ patients was higher than what was expected (19.6% vs 10.3%, respectively). The observed percentage of p/p patients was lower than what was expected (28.3% vs 43.3% respectively).

TABLE 30
Expected Genotype Distribution vs Observed Distribution
(Overall Male Cohort (n = 52) and Positive
Male Responders Only (n = 31))

| | Expected Genotype | Cohort Stratified | Positive Responders |
|---|---|---|---|
| | Distribution | by Genotype(%) | Stratified by |
| | (Racially Weighted | ALL patients | Genotype (%) |
| Genotype | to Cohort) | (N = 52) | (N = 31) |
| +/+ | 10.3% | 17.3% (9/52) | 19.4% (6/31) |
| +/p | 46.3% | 38.5% (20/52) | 32.3% (10/31) |
| +/+ or +/p | 56.6% | 55.8% (29/52) | 51.6% (16/31) |
| p/p | 43.3% | 32.7% (17/52) | 35.5% (11/31) |

Results: In the overall male cohort (n=52), 17.3% of the patients were +/+, 38.5% were +/p, and 32.7% were p/p. The observed percentage of the p/p genotype was lower than expected (32.7% vs 43.3%, respectively). The observed percentage of +/+ genotype was higher than expected (17.3% vs 10.3% respectively).

In the cohort of male positive responders (n=31), 19.4% of the patients were +/+, 32.3% were +/p, and 35.5% were p/p. The observed percentage of the p/p genotype was lower than expected (35.5% vs 43.3%, respectively). The observed percentage of the +/+ genotype was higher than expected (19.4% vs 10.3%, respectively).

TABLE 31
Expected Genotype Distribution vs Observed Distribution
(Overall Female Cohort (n = 26) and Positive Male
Responders Only (n = 15))

| | Expected Genotype | Cohort Stratified | Positive Responders |
|---|---|---|---|
| | Distribution | by Genotype(%) | Stratified by |
| | (Racially Weighted | ALL patients | Genotype (%) |
| Genotype | to Cohort) | (N = 26) | (N = 15) |
| +/+ | 10.3% | 11.5% (3/26) | 20.0% (3/15) |
| +/p | 46.3% | 53.8% (14/26) | 60.0% (9/15) |
| +/+ or +/p | 56.6% | 65.4% (17/26) | 80.0% (12/15) |
| p/p | 43.3% | 26.9% (7/26) | 13.3% (2/15) |

Results: In the overall female cohort (n=26), 11.5% of the patients were +/+, % were +/p, and 26.9% were p/p. The observed percentage of the p/p genotype was lower than expected (26.9% vs 43.3%, respectively). The observed percentage of +/+ genotype was higher than expected (11.5% vs 10.3% respectively).

In the cohort of female positive responders (n=15), 20.0% of the patients were +/+, 60.0% were +/p, and 13.3% were p/p. The observed percentage of the p/p genotype was lower than expected (13.3% vs 43.3%, respectively). The observed percentage of the +/+ genotype was higher than expected (20.0% vs 10.3%, respectively).

Several conclusions can be drawn from this study. Specifically, the female cohort provides the strongest support for these conclusions. First, patients lacking the active alleles, the p/p patients, respond poorer to therapy than patients with the active allele, the +/+ and +/p patients. For example, the female patients without the active allele respond 70% less frequently than the expected population to therapy, e.g., indoximod. Additionally, patients with the active allele respond better than those without. In the female cohort, patients with the +/+ or +/p alleles respond 41% more frequently than the expected population.

Each patent, patent application, and publication, including websites cited throughout specification are incorporated herein by reference. Similarly, the SEQ ID NOs which are referenced herein, and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### REFERENCES

1. Lee B, et al. Emerging biomarkers for immunomodulatory cancer treatment of upper gastrointestinal, pancreatic and hepatic cancers. Semin Cancer Biol 2018; 52:241-252.

2. Prendergast G C, et al. Indoleamine 2,3-dioxygenase pathways of pathogenic inflammation and immune escape in cancer. Cancer Immunol Immunother 2014; 63:721-35.

3. Munn D H, Mellor A L. IDO in the Tumor Microenvironment: Inflammation, Counter-Regulation, and Tolerance. Trends Immunol 2016; 37:193-207.

4. Theate I, et al. Extensive profiling of the expression of the indoleamine 2,3-dioxygenase 1 protein in normal and tumoral human tissues. Cancer Immunol Res 2015; 3:161-72.

5. Buque A, et al. Trial Watch—Small molecules targeting the immunological tumor microenvironment for cancer therapy. Oncoimmunology 2016; 5:e1149674.

6. Muller A J, et al. Inhibition of indoleamine 2,3-dioxygenase, an immunomodulatory target of the tumor suppressor gene Binl, potentiates cancer chemotherapy. Nat Med 2005; 11:312-9.

7. Monjazeb A M, et al. Blocking indolamine-2,3-dioxygenase rebound immune suppression boosts antitumor effects of radio-immunotherapy in murine models and spontaneous canine malignancies. Clin Cancer Res 2016; 22:4328-40.

8. Holmgaard R B,. Indoleamine 2,3-dioxygenase is a critical resistance mechanism in antitumor T cell immunotherapy targeting CTLA-4. J Exp Med 2013; 210:1389-402.

9. Merlo L M, et al. IDO2 Is a critical mediator of autoantibody production and inflammatory pathogenesis in a mouse model of autoimmune arthritis J Immunol 2014; 92:2082-90.

10. Merlo L M, et al., IDO2 Modulates T cell-dependent autoimmune responses through a B cell-intrinsic mechanism. J Immunol 2016; 196:4487-97.

11. Metz R, et al. IDO2 is critical for IDO1-mediated T cell regulation and exerts a non-redundant function in inflammation. Int Immunol 2014; 26:357-67

12. Vogel C F, et al. Aryl hydrocarbon receptor signaling mediates expression of indoleamine 2,3-dioxygenase. Biochem Biophys Res Commun 2008; 375:331-5.

13. Trabanelli S, et al. The SOCS3-independent expression of IDO2 supports the homeostatic generation of T regulatory cells by human dendritic cells J Immunol 2014; 192:1231-40.

14. Witkiewicz A K, et al. Genotyping and expression analysis of IDO2 in human pancreatic cancer: a novel, active target. J Am Coll Surg 2009; 208:781-7;

15. Witkiewicz A, et al. Expression of indoleamine 2,3-dioxygenase in metastatic pancreatic ductal adenocarcinoma recruits regulatory T cells to avoid immune detection. J Am Coll Surg 2008; 206:849-54;

16. Metz R, et al. Novel tryptophan catabolic enzyme IDO2 is the preferred biochemical target of the antitumor indoleamine 2,3-dioxygenase inhibitory compound D-1-methyl-tryptophan. Cancer Res 2007; 67:7082-7.

17. Hingorani S R, et al. Preinvasive and invasive ductal pancreatic cancer and its early detection in the mouse. Cancer Cell 2003; 4:437-50.

18. Gunderson A J, et al. Bruton tyrosine kinase-dependent immune cell cross-talk drives pancreas cancer. Cancer Discov 2016; 6:270-85.

19. Cook N, et al. K-Ras-driven pancreatic cancer mouse model for anticancer inhibitor analyses. Methods Enzymol 2008; 439:73-85.

20. Bush A, et al. c-myc null cells misregulate cad and gadd45 but not other proposed c-Myc targets. Genes Dev 1998; 12:3797-802.

21. Norris A L, et al. Familial and sporadic pancreatic cancer share the same molecular pathogenesis. Fam Cancer 2015; 14:95-103.

22. Garte S J. The c-myc oncogene in tumor progression. Crit Rev Oncog 1993; 4:435-49.

23. Fay M P, Shaw P A. Exact and asymptotic weighted logrank tests for interval censored data: the interval R package. J Stat Softw 2010; 36:i02.

24. Gao J, et al. Integrative analysis of complex cancer genomics and clinical profiles using the cBioPortal. Sci Signal 2013; 6:p11.

25. Cerami E, et al. The cBio cancer genomics portal: an open platform for exploring multidimensional cancer genomics data. Cancer Discov 2012; 2:401-4.

26. Becht E, et al. Estimating the population abundance of tissue-infiltrating immune and stromal cell populations using gene expression. Genome Biol 2016; 17:218.

27. Becht E, et al Immune and stromal classification of colorectal cancer is associated with molecular subtypes and relevant for precision immunotherapy. Clin Cancer Res 2016; 22:4057-66.

28. Hruban R H, et al. Progression model for pancreatic cancer. Clin Cancer Res 2000; 6:2969-72.

29. Chang H H, et al. Incidence of pancreatic cancer is dramatically increased by a high fat, high calorie diet in KrasG12D mice. PLoS One 2017; 12:e0184455.

30. Nevler A, et al. A sub-type of familial pancreatic cancer: evidence and implications of loss-of-function polymorphisms in indoleamine-2,3-dioxygenase-2. J Am Coll Surg 2018; 226:596-603.

31. Eldredge H B, et al. Concurrent whole brain radiotherapy and short-course chloroquine in patients with brain metastases: a pilot trial. J Radiat Oncol 2013; 2:315-21.

32. Jones W E 3rd., et al. ACR Appropriateness criteria(R) resectable pancreatic cancer. Am J Clin Oncol 2017; 40:109-17.

33. Alagappan M, et al. Albumin and neutrophil-lymphocyte ratio (NLR) predict survival in patients with pancreatic adenocarcinoma treated with SBRT. Am J Clin Oncol 2018; 41:242-247.

34. van Baren N, Van den Eynde B J. Tumoral Immune resistance mediated by enzymes that degrade tryptophan. Cancer Immunol Res 2015; 3:978-85.

35. Lob S, et al. IDO1 and IDO2 are expressed in human tumors: levo- but not dextro-1-methyl tryptophan inhibits tryptophan catabolism. Cancer Immunol Immunother 2009; 58:153-7.

36. Wang W, et al. IDO immune status after chemoradiation may predict survival in lung cancer patients. Cancer Res 2018; 78:809-16.

37. Prendergast G C, et al. Discovery of IDO1 inhibitors: from bench to bedside. Cancer Res 2017; 77:6795-811.

38. Lee K E, et al. Hif1a deletion reveals pro-neoplastic function of B cells in pancreatic neoplasia. Cancer Discov 2016; 6:256-69.

39. Oettle H, et al. Adjuvant chemotherapy with gemcitabine and long-term outcomes among patients with resected pancreatic cancer: the CONKO-001 randomized trial. JAMA 2013; 310:1473-81.

40. Neoptolemos J P, et al. Adjuvant chemotherapy with fluorouracil plus folinic acid vs gemcitabine following pancreatic cancer resection: a randomized controlled trial. JAMA 2010; 304:1073-81.

41. Valle J W, et al. Optimal duration and timing of adjuvant chemotherapy after definitive surgery for ductal adenocarcinoma of the pancreas: ongoing lessons from the ESPAC-3 study. J Clin Oncol 2014; 32:504-12.

42. Neoptolemos J P, et al. Comparison of adjuvant gemcitabine and capecitabine with gemcitabine monotherapy in patients with resected pancreatic cancer (ESPAC-4): a multicentre, open-label, randomised, phase 3 trial. Lancet 2017; 389:1011-24.

43. Morganti A G, et al. Multi-institutional pooled analysis on adjuvant chemoradiation in pancreatic cancer. Int J Radiat Oncol Biol Phys 2014; 90:911-7.

44. Sugawara A, Kunieda E. Effect of adjuvant radiotherapy on survival in resected pancreatic cancer: a propensity score surveillance, epidemiology, and end results database analysis. J Surg Oncol 2014; 110:960-6.

45. Lim Y J, et al. Role of adjuvant radiotherapy in left-sided pancreatic cancer-population-based analysis with propensity score matching. J Gastrointest Surg 2015; 19:2183-91.

46. Patel A A, et al. Early vs. late chemoradiation therapy and the postoperative interval to adjuvant therapy do not correspond to local recurrence in resected pancreatic cancer. Pancreat Disord Ther 2015; 5. pii: 151.

47. Cloyd J M, et al. Impact of hypofractionated and standard fractionated chemoradiation before pancreatoduodenectomy for pancreatic ductal adenocarcinoma. Cancer 2016; 122:2671-9.

48. Neoptolemos J P, et al. A randomized trial of chemoradiotherapy and chemotherapy after resection of pancreatic cancer. N Engl J Med 2004; 350:1200-10.

49. Prendergast G C, et al. Discovery of IDO1 inhibitors: from bench to bedside. Cancer Res 2017; 77:6795-6811.

50. Hou D Y, et al. Inhibition of indoleamine 2,3-dioxygenase in dendritic cells by stereoisomers of 1-methyl-tryptophan correlates with antitumor responses. Cancer Res 2007; 67:792-801.

51. Nevler A. et al., Host IDO2 Gene Status Influences Tumor Progression and Radiotherapy Response in KRAS-Driven Sporadic Pancreatic Cancers. Clin. Cancer Res., Jan. 2019, 25(2):724-734 (pub. Online Sept 28, 2018)

1. A method of predicting the responsiveness of a subject having a disease to a treatment regimen comprising performing a genotype assay to determine the presence, absence or mutation of least one of the Indoleamine 2,3-dioxygenase 2 (IDO2) gene alleles at the single nucleotide polymorphism (SNP) site rs4503084 or the SNP site rs10109853.

2. The method according to claim 1, further comprising:
(a) obtaining DNA from a biological sample of said subject;
(b) contacting the DNA sample from the subject with reagents to determine the presence, absence or a mutation at the following wildtype alleles:
i. CC at single nucleotide polymorphism (SNP) site rs4503084
ii. TT at SNP site rs10109853.

3. The method according to claim 1, wherein said treatment regimen involves treatment with an inhibitor of Indoleamine 2,3-dioxygenase 2 (IDO), an inhibitor of tryptophan 2,3-dioxygenase (TDO), or an inhibitor of the IDO/TDO pathway.

4. The method according to claim 3, wherein subjects with at least one wildtype allele are more likely to respond positively to treatment with and IDO or TDO inhibitor than subjects lacking active alleles.

5. The method according to claim 3, wherein subjects with a mutation at both alleles are less likely to respond to treatment with IDO or TDO inhibitor than subjects with wildtype alleles.

6. (canceled).

7. The method according to claim 1, wherein said treatment regimen involves a combination of treatment with an inhibitor of Indoleamine 2,3-dioxygenase 2 (IDO), an inhibitor of tryptophan 2,3-dioxygenase (TDO), or an inhibitor of the IDO/TDO pathway with a second therapy.

8. The method according to claim 7, wherein the second therapy is a radiotherapy, chemotherapy or immunotherapy.

9. (canceled)

10. The method according to claim 1, wherein the immunotherapy comprises the administration of an anti-PD1 composition or dendritic cell therapy.

11. (canceled)

12. The method according to claim 1, wherein the disease is a cancer.

13. The method according to claim 12, wherein the cancer is pancreatic cancer or pancreatic ductal adenocarcinoma (PDAC), melanoma, breast cancer, brain cancer, colon/rectal cancer, lung cancer, ovarian cancer, adrenal cancer, anal cancer, bile duct cancer, bladder cancer, bone cancer, endometrial cancer, esophagus cancer, eye cancer, kidney cancer, laryngeal cancer, liver cancer, head and neck cancer, nasopharyngeal cancer, osteosarcoma, oral cancer, ovarian cancer, prostate cancer, rhabdomyosarcoma, salivary gland cancer, stomach cancer, testicular cancer, thyroid cancer, vaginal cancer, lung cancer, and neuroendocrine cancer, or glioblastoma.

14. The method according to claim 1, wherein the disease is chronic infection, an autoimmune disease, or retinopathy.

15. The method according to claim 1, wherein the occurrence of a biallelic inactivating mutation at either or both said SNP sites indicates that the subject would have a positive response to said treatment involving radiotherapy.

16. The method according to claim 1, wherein the subject's genotype showing inactivating SNP site mutations on a single allele indicates that the subject would have some response to said treatment.

17. The method according to claim 1, wherein the subject's genotype showing wild-type nucleotides at both SNP sites indicates that the subject would have a stronger positive response to said treatment involving chemotherapy.

18. The method according to claim 8, wherein the disease is cancer and the second therapy is radiation therapy and the inhibitor is indoximod or chloroquine.

19. The method according to claim 1 wherein subject's demonstrating biallelic mutation at the two SNP sites indicates that said subject is more responsive to radiotherapy.

20. The method according to claim 8, wherein the disease is cancer, the chemotherapeutic is gemcitabine and the IDO inhibitor is indoximod.

21. A method of assessing the risk of a subject for the onset or progression of cancer comprising performing a genotype assay to determine the presence, absence or mutation of the Indoleamine 2,3-dioxygenase 2 (IDO2) gene at the single nucleotide polymorphism (SNP) site rs4503084 and the SNP site rs10109853, wherein the presence of a mutation at one or both said SNP sites that inactivates the IDO2 activity of both alleles indicates that said subject has a decreased risk of cancer onset.

22. The method according to claim 13, wherein the subject's cancer is pancreatic cancer.

23. The method according to claim 13, wherein the subject's cancer is melanoma.


SEQUENCE LISTING (5 sequences)

SEQ ID NO: 1 - DNA, 21 bp, Homo sapiens
gaacattctatcccccgttgc

SEQ ID NO: 2 - DNA, 24 bp, Homo sapiens
ttacctgagagtggatccctagca

SEQ ID NO: 3 - DNA, 21 bp, Homo sapiens
tcttgtgctccctccaaaaca

SEQ ID NO: 4 - DNA, 20 bp, Homo sapiens
tggtttggcttcccatgctt

SEQ ID NO: 5 - protein, 420 aa, Homo sapiens
MLHFHYYDTSNKIMEPHRPNVKTAVPLSLESYHISEEYGPLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGNLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHPRG